(19) United States
(12) Patent Application Publication
(10) Pub. No.: US 2022/0296758 A1
(43) Pub. Date: Sep. 22, 2022

(54) CHEMICAL-FREE MOIST HEAT DECONTAMINATION UNIT AND METHOD

(71) Applicant: Crescent Metal Products, Inc., Mentor, OH (US)

(72) Inventors: Adolph DeGennaro, Fairport Harbor, OH (US); Yogesh Ramadoss, Cleveland Heights, OH (US); Gregory Baggott, Chardon, OH (US)

(21) Appl. No.: 17/696,296

(22) Filed: Mar. 16, 2022

Related U.S. Application Data
(60) Provisional application No. 63/162,112, filed on Mar. 17, 2021.

(51) Int. Cl. A61L 2/26; A61L 2/07; A61L 2/24

(52) U.S. Cl. CPC A61L 2/26; A61L 2/07; A61L 2/24

(57) ABSTRACT

A decontamination unit includes a housing having an internal cavity for containing a contaminated object that is to be decontaminated, a heater in thermal communication with the internal cavity and is configured to heat the internal cavity and object, and a humidity generator in fluid communication with the internal cavity and configured to supply humidity to the internal cavity. A controller is configured to control a temperature and a relative humidity within the internal cavity according to a decontamination cycle for decontaminating the object. The decontamination cycle may be preset based on the object(s) and/or type of contaminant. According to one aspect, the decontamination cycle is performed with at least the parameters of a temperature in a range from 170° F. to 200° F., a relative humidity in a range from 50% to 80%, and a time in a range from 15 minutes to 60 minutes.

### RELATED APPLICATIONS

This application claims the benefit of U.S. Provisional Application No. 63/162,112 filed Mar. 17, 2021, which is hereby incorporated herein by reference in its entirety.

### TECHNICAL FIELD

The present invention relates generally to decontamination units, and more particularly, to a chemical-free moist heat decontamination unit and/or chemical-free most heat decontamination method.

### BACKGROUND

Decontamination units generally involve a process of eliminating contaminants from an object, such as the destroying or removal of micro-organisms, viruses, bacteria, or the like, some of which may be harmful to human health. There are various types of decontamination units depending on the contaminant to be removed.

### SUMMARY

Using moist heat in the form of steam is one conventional approach to decontaminating or sterilizing objects. Conventionally this involves the use of autoclaves that operate at higher pressures (e.g., in excess of 15 psi) and at high temperatures (e.g., in excess of about 120° C. (248° F.)) to generate the steam. These relatively harsh conditions (especially the elevated temperatures) can have a detrimental impact on the physical properties and functionality of the decontaminated object. For example, depending on the circumstances, it may be desirable to reuse disposable respirators employing fabrics and elastic bands, which these materials may degrade with conventional moist heat decontamination techniques using high temperature. In addition, because of the temperatures and pressures involved, conventional autoclaves can be complex and costly pieces of equipment that are not readily available at the point of use of the contaminated article to be sterilized.

Furthermore, sterilizers used to sterilize objects may utilize pressurized chambers and/or vacuum chamber. Additionally, sterilizers may include HEPA filters which contain filtration systems and additional components. Sterilizers may further include air intakes and/or effluent management systems. These features make sterilizers costly to manufacture and use for decontamination of objects.

According to an aspect, a moist heat decontamination unit and method is provided herein that overcomes one or more of the foregoing deficiencies associate with conventional autoclaves or other moist heat decontamination techniques.

More particularly, according to an aspect, a unique moist heat decontamination unit and method is provided herein that includes one or more advantages of: (i) a relatively low-temperature decontamination cycle with an associated relative humidity for decontaminating an object; (ii) decontamination at atmospheric pressure; (iii) decontamination without the use of chemicals; (iv) a relatively short cycle time for increasing decontamination throughput of contaminated objects, which may be particularly advantageous in the event of a catastrophe or pandemic, for example; (v) a relatively inexpensive unit that is affordable and deployable at various points of use, such as at schools, police stations, fire stations, department of defense, other government agencies, elderly care facilities, hospitals, and the like; and/or (vi) an enhanced modularity or tailorability of the internal object-support or racking system design for enabling decontamination of many different types of objects, such as difficult to wash first responder service wear (e.g., paramedic, police, or firefighter protective gear, including fire jackets, fire trousers, fire gloves, fire helmets, ballistic vests) or the like, model 1860 N95 (3M, Minneapolis, USA) respirators and other masks, school supplies, or the like.

According to an aspect, a moist heat decontamination unit is configured to sufficiently decontaminate an object based on the object to be decontaminated and/or the contaminant on the object.

For example, a decontamination cycle of the unit may provide a >6 log reduction of the contaminant, such as MS2 Bacteriophage (non-lipid surrogate for Sars-Cov-2), Mycobacteria, C. difficile spores, or equivalently difficult to eliminate bacteria, viruses, spores, or the like.

On the other hand, the decontamination unit may be specifically adapted to be cost-effective and robust enough to provide a repeatable time, temperature, and humidity cycle that can significantly reduce certain bacterial and viral load(s), but the unit is not so complex as to significantly reduce other more difficult to eliminate viruses and bacteria. For example, the exemplary cost-effective decontamination unit may be specifically adapted with a time, temperature, and humidity profile that provides at least between a 3 log and 7 log reduction in viral or bacterial loads, including but not limited to, enveloped virus (coronavirus, influenza, HIV, rubella), vegetative bacteria (Salmonella, E. coli, S. aureus, Pseudomonas species), fungi (Aspergillus species, Candida species), non-enveloped viruses (MS2 bacteriophage, rotavirus, norovirus), mycobacteria (Mycobacterium species), or equivalents. However, other more difficult to eliminate bacteria and viruses such as bacterial spores (Bacillus species (Bacillus subtilis), Clostridium species, C. difficile spores), or equivalents are not intended to be significantly reduced (e.g., less than 3 log reduction) by the exemplary decontamination unit, as this may otherwise require a relatively high temperature and humidity (or excessive pressure/vacuum) that is difficult to maintain or otherwise may demand a greater complexity that is not a cost-effective solution, such as for everyday use at first responder locations, schools, or the like.

According to an aspect, a moist heat decontamination unit includes: a housing having an internal cavity and an opening for accessing the internal cavity; a door coupled to the housing for opening or closing the opening for permitting or restricting access to the internal cavity; a heater in thermal communication with the internal cavity and configured to heat the internal cavity; a humidity generator in fluid communication with the internal cavity and configured to supply humidity to the internal cavity; a controller operatively coupled to at least the heater, the controller being configured to control a temperature and a relative humidity within the internal cavity according to a preset decontamination cycle having at least the following parameters: the temperature of the preset decontamination cycle is in a range from 170° F. to 200° F.; the relative humidity of the preset decontamination cycle is in a range from 50% to 80%; and a time of the preset decontamination cycle is in a range from 15 minutes to 60 minutes.

According to an aspect, a method of decontaminating an object includes: placing the object within an internal cavity of a decontamination unit; supplying heat to the internal cavity and increasing the temperature of the internal cavity to a temperature value in a range from 170° F. to 200° F.; supplying humidity to the internal cavity and increasing the relative humidity of the internal cavity to a relative humidity value in a range from 50% to 80%; and performing decontamination at the temperature value and at the relative humidity value for a period of time in a range from 15 minutes to 60 minutes.

According to an aspect, a moist heat decontamination unit includes racking or other suitable supports that are adapted for holding first responder equipment in the cabinet. This may include, for example, suitable hanging rods for hanging jackets, trousers vests, or the like via loops on the garments or via a suitable hanger. Such racking also may include adjustable racks for holding helmets, gloves, or other protective equipment.

According to another aspect, a method of decontaminating first responder equipment includes: (i) placing the first responder equipment within an internal cavity of a decontamination unit; supplying heat to the internal cavity; and supplying humidity to the internal cavity for a time; wherein the supplying heat and the supplying humidity is effective to provide at least between a 3 log to 7 log reduction in bacterial or viral load. The method may be effective to provide such a reduction in enveloped viruses, vegetative bacteria, fungi, non-enveloped viruses, mycobacteria (Mycobacterium species), or the like. The first responder equipment may include jackets, vests, gloves, and/or helmets.

The following description and the annexed drawings set forth certain illustrative embodiments of the invention. These embodiments are indicative, however, of but a few of the various ways in which the principles of the invention may be employed. Other objects, advantages and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention.

FIG. 1 is a front elevational view of an exemplary moist heat decontamination unit according to an embodiment with doors removed.

FIG. 2 is a side elevational view of the decontamination unit.

FIG. 3 is a perspective front view of the decontamination unit with an upper door removed.

FIG. 4 is an enlarged view of section “A” shown in FIG. 3.

FIG. 5 shows an exemplary shelving unit for use in the decontamination unit.

FIG. 6 shows one exemplary form of a support in the decontamination unit for disposable respirator masks.

FIG. 7 shows another exemplary form of an object support system in the decontamination unit for firefighter gear.

FIG. 8 is a perspective view of the decontamination unit with an outer shell and doors removed showing an exemplary air flow of the unit schematically.

FIG. 9 is an exemplary schematic view of the decontamination unit shown in FIG. 1.

FIG. 10 is a flow chart showing an exemplary decontamination process according to an embodiment.

FIG. 11 is an infographic showing resistance to kill and level of kill for common bacteria and viruses.

FIGS. 12 and 13 are bar charts depicting the data in Table 1.

FIGS. 14 and 15 are bar charts depicting the data in Table 2.

FIGS. 16 and 17 are bar charts depicting the data in Table 3.

FIGS. 18 and 19 are bar charts depicting the data in Table 4.

### DETAILED DESCRIPTION

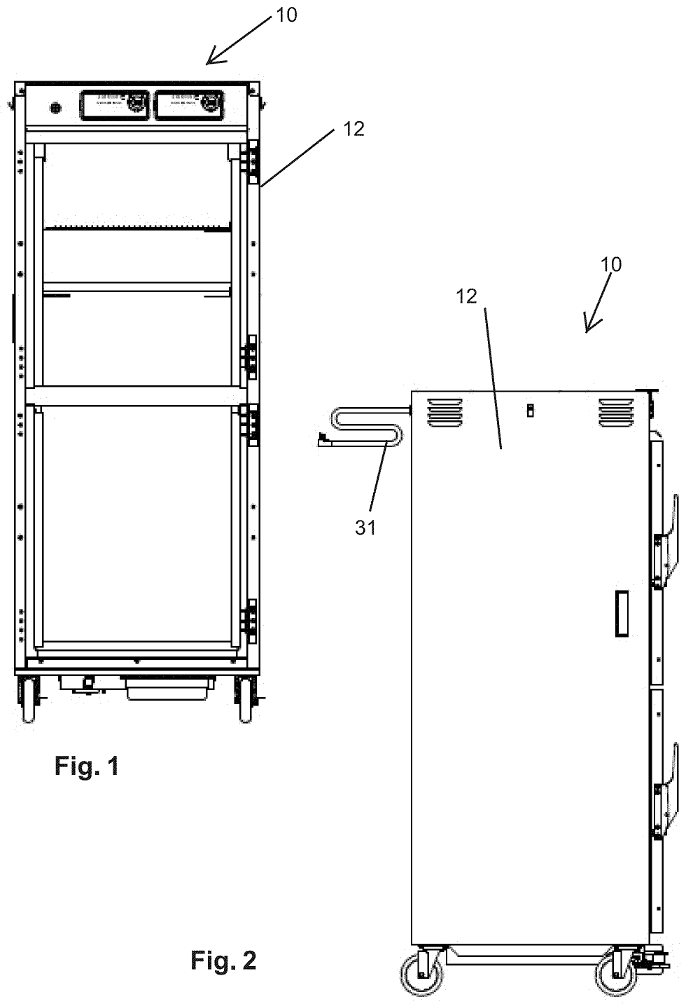

Referring to the drawings, and initially to FIGS. 1-4, 8 and 9, an exemplary moist heat decontamination unit 10 is shown. The decontamination unit 10 includes a housing 12 having an internal cavity 14 for containing a contaminated object 15 that is to be decontaminated. The decontamination unit 10 also includes a heater 16 that is in thermal communication with the internal cavity 14 and is configured to heat the internal cavity 14 and object 15, and also a humidity generator 18 that is in fluid communication with the internal cavity 14 and configured to supply humidity to the internal cavity 14 that contacts the object 15. A controller 20 is provided that is configured to control a temperature and a humidity within the internal cavity 14 according to a decontamination cycle for decontaminating the object 15. The decontamination cycle may be performed at atmospheric pressure. In exemplary embodiments, the decontamination cycle may be performed free from the use of chemicals.

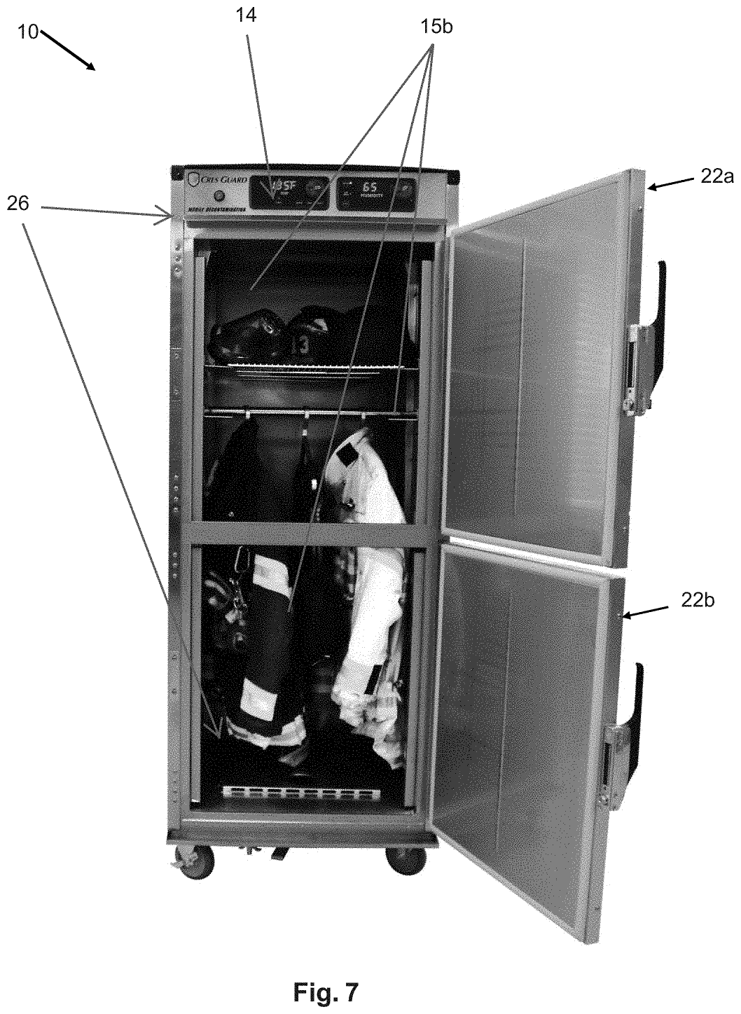

In exemplary embodiments, the housing 12 includes a base and a main body, which may be composed of one or more parts. The housing 12 may be reinforced by a frame, such as an internal stainless-steel frame, and may be made of any suitable material or combination of materials. One or more doors 22a, 22b (see FIG. 7) are provided for opening or closing an opening 23 or openings in the front of the housing 12 for allowing access to the internal cavity 14.

The housing 12 and the door(s) 22a, 22b may be insulated to maintain the desired temperature range inside of the cavity 14 and/or prevent temperature loss. The housing 12 may include an outer housing portion, which forms an outer surface of the housing, and an inner housing portion, which forms an inner surface of the internal cavity 14 (also referred to as internal chamber 14). The outer housing portion may be made of any suitable material, such as powder-coated aluminum. The inner housing portion may be made of any suitable material, such as stainless steel, and may include smooth interior coved corners to prevent buildup of material. An insulation gap may be formed between the outer housing portion and the inner housing portion, in which the insulation gap may be filled with an insulation material, such as fiberglass and/or foam. The doors 22a, 22b may be made of stainless steel and may include a gasket such as a magnetic santoprene.

The door(s) 22a, 22b are coupled to the housing 12 by any suitable means, such as by hinges. The door(s) 22a, 22b each include a handle configured to latch to the housing 12 by a suitable latch, such as a magnetic latch. The door can be equipped with any suitable lock to lock the decontamination unit 10, for example, during transportation or during a decontamination cycle. One or more of the door(s) 22a, 22b also may include a viewing window, which may be made of any suitable material, such as glass, acrylic glass, etc.

In exemplary embodiments, the decontamination unit 10 (also referred to as a cabinet, or simply unit 10) may be configured to be portable. In the illustrated embodiment, for example, a plurality of wheels 24 are provided at a bottom side of the base that allow the decontamination unit 10 to be moved easily, even when fully loaded. A brake may be provided on one or more of the wheels 24 so that the cabinet can be locked in place when being used. In the illustrated embodiment, the front two wheels are swivel casters with brakes, and the rear two wheels are fixed to provide mobility when fully loaded.

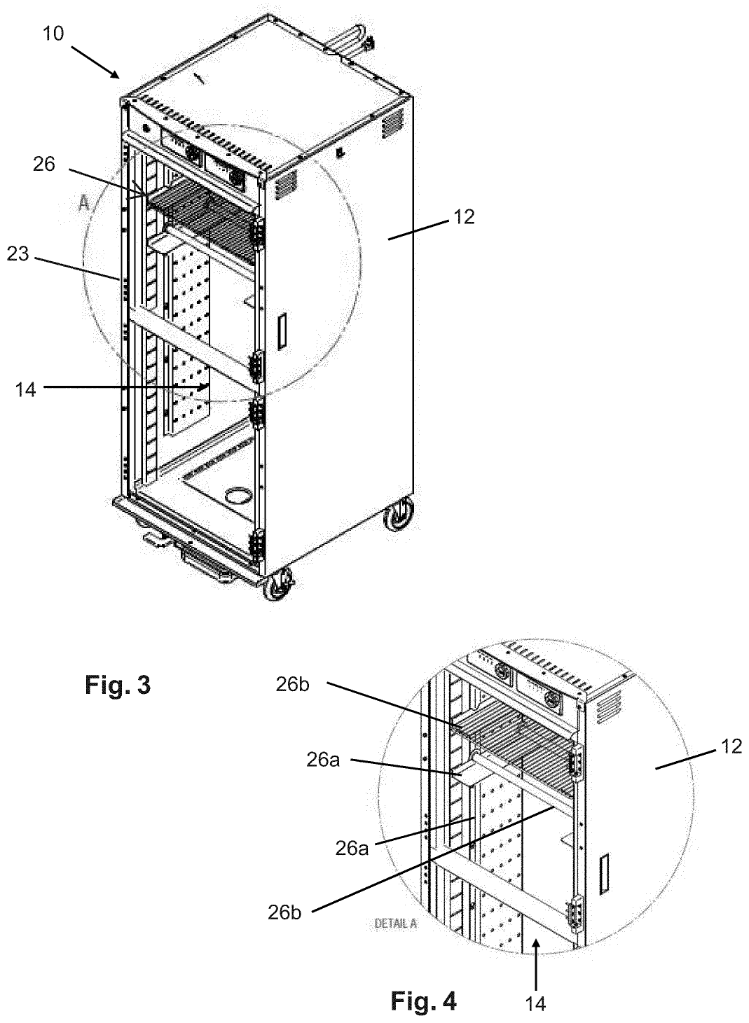

Referring particularly to FIGS. 3-5, the decontamination unit 10 is shown with the doors 22a, 22b removed. As shown, the unit 10 may include any suitable type or amount of object supports 26 in any suitable configuration for holding the objects to be decontaminated. For example, in the illustrated embodiment, the decontamination unit 10 may include vertically spaced apart pairs of supports 26a, such as rails, slots, holes, or angles, within the internal cavity 14. Some of the pairs of supports 26a may also be laterally spaced apart to provide rows of supports 26a, for example, rows of holes for accepting shelves. The pairs of supports 26a support laterally extending supports 26b in the form of shelves and/or trays. In other embodiments, the supports 26b may be in the form of laterally extending rods and/or bars for hanging straps or clothes, such as, but not limited to, coats. In yet another embodiment, the supports 26 may be a combination of shelves and/or trays and rods and/or bars. The supports 26 may be arranged vertically spaced apart from each other in the internal cavity 14. Alternatively, the supports 26 may be laterally spaced apart from each other. In exemplary embodiments, the various supports 26 can be adjusted and configured in any suitable manner to handle smaller objects and/or bulky items.

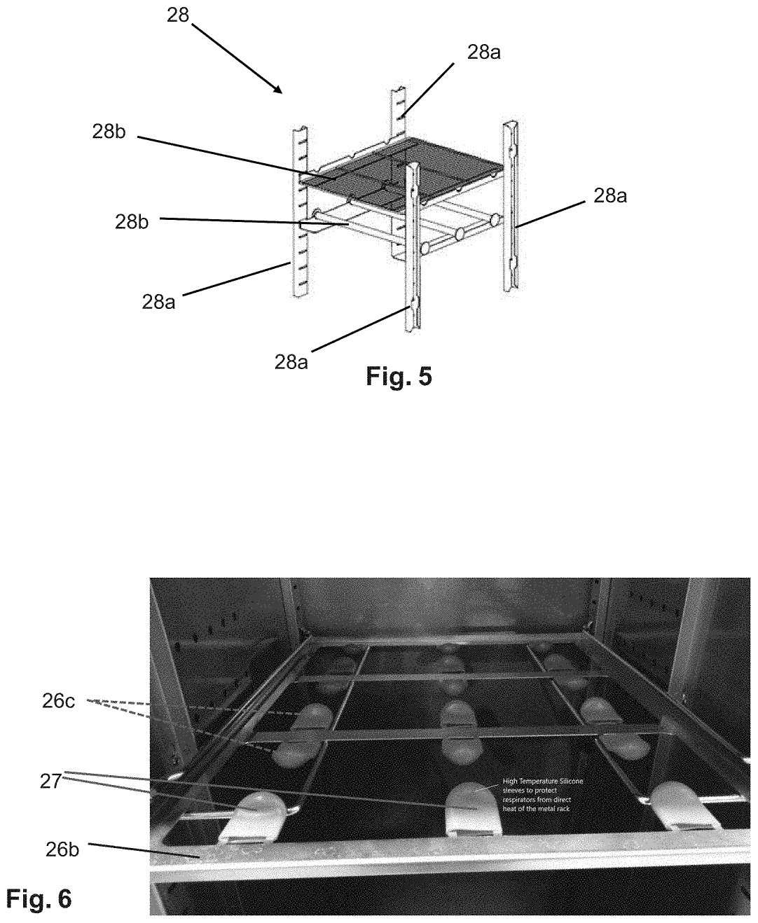

With reference specifically to FIG. 5, at least one shelving unit 28 may be assembled for inserting into the internal cavity. The shelving unit 28 may include any configuration necessary to accommodate the desired objects being decontaminated. The shelving unit 28 may include a plurality of vertical racks 28a having slots for supporting laterally extending supports 28b in the form of shelves and/or trays. In another embodiment, the supports 28b may be in the form of laterally extending rods and/or bars for hanging straps or clothes, such as, but not limited to, coats. In yet another embodiment, as shown in FIG. 5, the supports 28b may comprise a combination of shelves or trays and rods or bars. One or more shelving units 28 may be stacked above each other in the internal cavity 14.

FIG. 6 shows one exemplary form of a support 26 for disposable N95 respirator masks 15a (made by 3M Corporation, Minneapolis Minn.). As shown in the figures, the laterally extending support 26b includes a plurality of horizontal projections 26c, or prongs, (also shown in FIG. 5) that are covered with high-temperature silicone sleeves 27. This enables the masks 15a to be hanged by their elastic straps, without excessive temperature conduction through the silicon sleeves 27 that would exceed the thermal degradation temperature of the elastic straps.

FIG. 7 shows another exemplary form of an object support system 26, or racking, that is configured to hold firefighter gear 15b. As shown, jackets, helmets or other bulky objects may be hanged by horizontal supports. A lower support (not shown) may hold boots, and an upper support may hold objects such as helmets and gloves. Such objects generally are too bulky to be sent through a conventional washing machine, whereas the exemplary decontamination unit 10 may use a decontamination cycle to decontaminate and clean the firefighter gear 15b. It is understood that that the racking/supports 26 may be modified as desired for holding other service wear for other applications, such as EMS uniforms, police uniforms, nursing uniforms, or other first responder wear, for example. Examples of first responder wear include, but is not limited to, jackets, vests, garments such as clothing, helmets, gloves, shoes, ballistic vests, or the like. Additionally, the racking/supports 26 may also be modified as desired for holding other objects requiring decontamination such as, but not limited to, model 1860 N95 (3M, Minneapolis, USA) respirators and other masks, select medical equipment, uniforms, or school supplies.

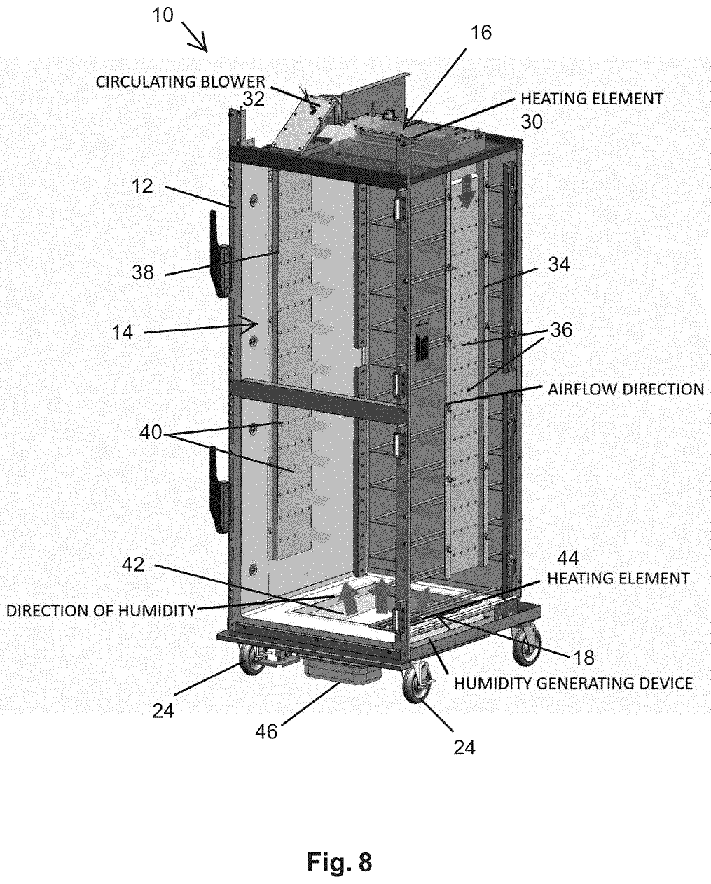

Referring particularly to FIG. 8, the heater 16 of the decontamination unit 10 and an exemplary heated air flow cycle are shown. The heater 16 may be any suitable heater capable of heating the internal cavity 14 as desired, which may include one or more of convection, conduction, and radiation heating. In the illustrated embodiment, the heater 16 includes an electric heating element 30 that is electrically coupled to a power source by an electrical cord 31 (FIG. 2). The electric heating element 30 may be any suitable heating element, such as a resistive heating element, for example. In exemplary embodiments, the heating element 30 is disposed in a cavity outside of the internal cavity 14 and heats the internal cavity 14 via convective heating. In the illustrated embodiment, for example, the heating element 30 is disposed in an upper cavity above the internal cavity.

To provide convective heating to the internal cavity 14 via the heating element 30, a blower 32 is configured to blow air across the heating element 30 and into the internal cavity 14. The blower 32 may be any suitable type of blower at any suitable location in the decontamination unit 10 for transferring heated air from around the heating element 30 into the internal cavity 14. As shown in the illustrated embodiment, for example, the blower 32 is disposed in the upper cavity where air from the blower blows across the heating element 30 and travels through a vertical supply channel 34, or air passage, where it is forced into the internal cavity 14 via openings (inlets) 36 in the vertical supply channel 34. On another side (e.g., opposite side) of the internal cavity 14, a vertical return channel 38, or air passage, is provided having a plurality of openings 40 (outlets) for drawing the air from the internal cavity 14 into the return channel 38. The blower 32 is operatively connected to the return channel 38 and sucks the return air back through the blower 30 and across the heating element 30. In this manner, the air within the decontamination unit 10 is recycled and is not exhausted until the door(s) 22a, 22b are opened. Suitable seals may be provided to substantially maintain the hot air within the unit 10.

Unlike sterilizers, however, the exemplary decontamination unit 10 does not comprise an air intake and/or effluent management systems. Furthermore, the exemplary decontamination unit 10 does not comprise any pressurized chambers or vacuum chambers. It is an advantage of the decontamination unit 10 to not include intake or effluent management systems(s), HEPA filtration, or any pressurized chambers or vacuum chambers, but instead can be used at atmospheric pressure. By not including these complex features, the exemplary decontamination unit 10 is cheaper to manufacture and use, unlike conventional sterilizers.

Still referring to FIG. 8, the humidity generator 18 of the decontamination unit 10 also is shown. The humidity generator 18 may be any suitable device that is capable of supplying humidity to the internal cavity 14 as may be desired for particular application(s). In exemplary embodiments, the humidity generator 18 includes a moisture source 42 and a heating element 44 configured to heat the moisture source 42 to generate humidity in the air. In the illustrated embodiment, for example, the moisture source 42 includes a reservoir (also 42) that is in fluid communication with the internal cavity 14, such as via an opening in the reservoir (as shown) or via suitable passages. The opening to the reservoir may be covered by a pan or other cover which may have holes or other passages formed therein.

The reservoir 42 may contain liquid water of any suitable type (e.g., filtered, distilled, deionized, etc.). The water may be prefilled with a predefined quantity of initial water, or the water may be piped into the reservoir via an external source, such as a spigot, for continuous and/or controlled flow during operation. In the illustrated embodiment, the heating element 44 is submerged in the water and may be temperature controlled by the controller 20 based upon a measured or calculated amount of humidity in the internal cavity 14. The heating element 44 of the humidity generator 18 may be controlled independently of the heating element 30 of the heater 16. Alternatively, the hot air from the heater 16 may cross over a surface of the water in the reservoir 42 to generate humidity.

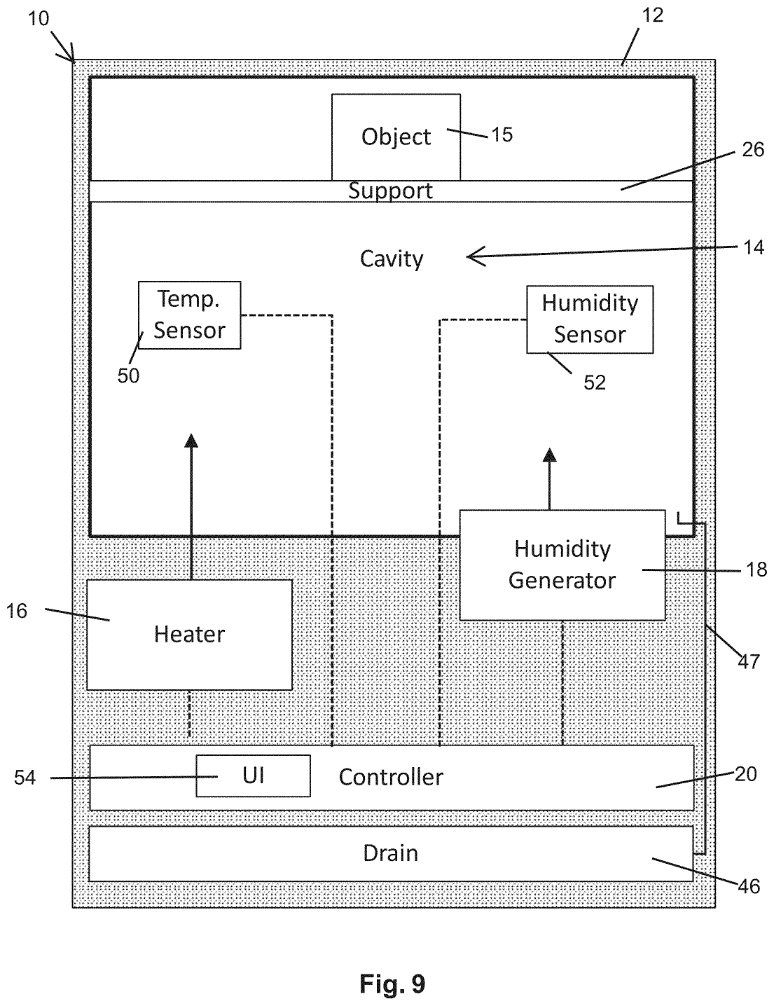

As shown, a drain 46 also may be provided for draining condensed water vapor from the chamber walls or other cooler surfaces within the internal cavity 14. The drain 46 may include a separate reservoir, such as a removable drain pan, or the drain may drain externally. Alternatively, condensation may be returned to the supply reservoir 42 or may be recycled from the drain to the supply reservoir. A drain line 47 (FIG. 9) is fluidly connected from the internal cavity 14 to the drain 46 in the illustrated embodiment. Unlike sterilizers, the decontamination unit 10, however, may not include a filtration system or similar components.

Turning to FIG. 9, a schematic illustration of the decontamination unit 10 is shown, including the internal cavity 14, at least one support 26 within the internal cavity 14 for holding the object(s) 15 to be decontaminated, the heater 16 in thermal communication with the internal cavity 14, and the humidity generator 18 in fluid (vapor) communication with the internal cavity 14. The drain 46 including the drain line 47 fluidly connected to the internal cavity 14 also is shown for draining condensation from the internal cavity.

As discussed above, a controller 20 is configured to control the temperature and humidity within the internal cavity 14 according to a decontamination cycle for decontaminating the object. In exemplary embodiments, the controller 20 is operatively coupled to at least the heater 16, and optionally to the humidity generator 18. As discussed above, for example, the heater 16 may include an electric heating element 30 and a blower 32, both of which may be controlled independently by the controller 20 to achieve the desired temperature in the cavity 14. Also as discussed above, the humidity generator 18 may include a heating element 44 submersed in or in close proximity to the moisture source 42 (e.g., water-filled reservoir 42), which also may be controlled independently by the controller 20 to achieve the desired relative humidity in the cavity 14. In embodiments that do not have a dedicated heat source for generating humidity (i.e., where heated air from the heater 16 is used to pass over the moisture source 42, for example), then the controller 20 may control the heating element 30 and/or blower 32 as needed to achieve the desired temperature and humidity parameters.

The controller 20 may include any suitable apparatus, device(s), or machine(s) for processing data, including a primary control circuit that is configured to carry out various control operations relating to control of the decontamination unit 10. The controller may include by way of example, a programmable processor, a computer, or multiple processors or computers. For example, the primary control circuit may include an electronic processor, such as a CPU, microcontroller or microprocessor. The operative connection(s) of the controller to the heater 16 and humidity generator 18 includes those in which signals, physical communications, or logical communications may be sent or received. Typically, an operative connection includes a physical interface, an electrical interface, or a data interface, but it is to be noted that an operative connection may include differing combinations of these or other types of connections sufficient to allow operative control.

The controller 20 also may include, in addition to hardware, code that creates an execution environment for the computer program in question. For example, among their functions, to implement the features described herein, the control circuit and/or electronic processor may comprise an electronic controller that may execute program code embodied as the decontamination unit control application. It will be apparent to a person having ordinary skill in the art of computer programming, and specifically in application programming for electronic and communication devices, how to program the device to operate and carry out logical functions and instructions associated with the control application. Accordingly, details as to specific programming code have been left out for the sake of brevity. The decontamination unit control application may be stored in a non-transitory computer readable medium, such as random access memory (RAM), a read-only memory (ROM), an erasable programmable read-only memory (EPROM or Flash memory), or any other suitable medium. It is understood that although instructions for performing the methods described herein may be executed by the processor components of the controller, such controller functionality could also be carried out via dedicated hardware, firmware, software, or combinations thereof.

To provide feedback control to the controller 20, one or more sensors for determining the temperature and relative humidity within the internal cavity also are provided and operatively coupled to the controller 20. Any suitable type or types of sensor(s) may be provided for communicating the measured temperature and humidity to the controller for providing control of these parameters. For example, in the illustrated embodiment, at least one temperature sensor 50 is provided in thermal communication with the internal cavity 14, such as a thermostat or thermocouple having a thermal sensing portion within the internal cavity 14. The temperature sensor 50, or another separate temperature sensor, also may be used for determining the relative humidity within the internal cavity by using a calculation and/or lookup table, for example. Alternatively or additionally, a separate humidity or moisture sensor 52 may be provided in suitable communication with the internal cavity 14 for measuring and communicating the relative humidity level within the cavity for humidity control by the controller 20.

The controller includes a user interface 54 that enables the user to start the decontamination cycle. Any suitable user interface may be provided, including one or more physical buttons, switches, knobs, sliders and the like; or an electrical touchscreen display with one or more of the foregoing. Suitable indicator(s) and/or displays, including lights, LCDs, LEDs, etc. also may be provided for displaying information, such as warning indicators, internal cavity temperature, internal cavity relative humidity, timer, and the like. The user interface 54 also may include an on/off switch to power on or power off the unit. In exemplary embodiments, the controller 20 may lock-out user interaction and interruption during a decontamination cycle.

The user interface 54 also may include a display for a menu of options to control the decontamination unit in a particular manner, such as selecting a desired decontamination cycle. For example, the controller 20 (or a non-transitory computer readable medium, such as a hard drive) may store a plurality of preset decontamination cycles depending on the objects to be decontaminated and/or the type of contaminant to be removed. Bulky items, such as firefighter gear, for example, may require a longer cycle due to the thermal mass involved in heating the internal cavity to the setpoint decontamination temperature. Viruses such as the coronavirus (e.g., sars-cov-2), or bacteria such as Staphylococcus aureus (e.g. MRSA), also may utilize different parameters for decontamination thereof.

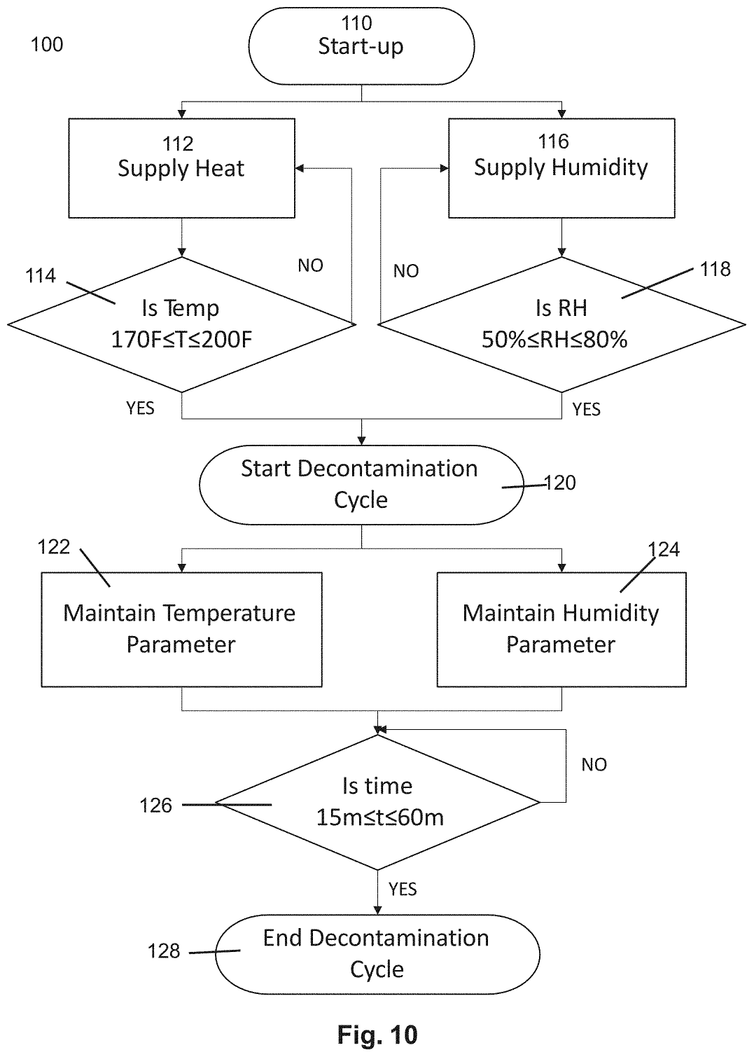

Based on experimentation with the decontamination unit 10, a decontamination cycle with the parameters and ranges identified in the exemplary flow chart of FIG. 10 are found to be effective in decontaminating contaminated objects. As noted above, the process may be performed at atmospheric pressure, with reasonably low temperatures, relatively short cycle times, and without the use of chemicals. In exemplary embodiments, the decontamination cycle is a preset cycle that runs a predetermined temperature profile, or setpoint value(s), a predetermined relative humidity profile, or setpoint value(s), and a predetermined period of time, as described in further detail below. As noted above, once a preset decontamination cycle is started, the controller 20 may lock-out the controls for preventing changing the temperature, humidity or time parameters.

As shown in FIG. 10, the process 100 begins at step 110 with a start-up of the decontamination unit 10, which may include powering the unit. At step 112, heat is supplied by the heater 16 to heat the internal cavity 14 to a setpoint temperature for the decontamination cycle. During this time, the temperature may ramp up at a predetermined ramp rate, or as fast as possible depending on the thermal load in the internal cavity 14 and power of the heater 16.

At step 114, in exemplary embodiments the setpoint temperature inside of the internal cavity 14 for the decontamination cycle is in a range from about 140° F. (60° C.) to about 220° F. (140° C.). In some preferred embodiments, the temperature inside of the internal cavity 14 is in a range from about 170° F. (77° C.) to about 200° F. (93° C.) In other embodiments, the temperature inside of the internal cavity 14 is in a range from about 165° F. (74° C.) to about 212° F. (100° C.). As such, the setpoint temperature for the decontamination cycle may be any value, range or subrange between or within the foregoing ranges, such as a temperature of about any of 140° F., 150° F., 160° F., 170° F., 180° F., 190° F., 200° F., 210° F., or 220° F. (including any values or subranges between the stated values). The lower temperatures may be particularly suitable for thermally sensitive objects, such as elastic bands, for example, that have a low thermal degradation temperature. The lower temperature setpoints also are faster and easier to achieve, and may enable the unit to provide more repeatable results with a less complex structure of the decontamination unit. Increased temperatures, however, may increase decontamination rate but may be at a cost of a more complex structure of the unit.

Step 116 shows supplying humidity to the internal cavity with the humidity generator 18. The supply of humidity to the internal cavity 14 at step 116 may occur substantially simultaneously with the supply of heat at step 112; or may be supplied consecutively after the temperature setpoint has been reached. During this time, the humidity may ramp up at a predetermined ramp rate, or as fast as possible.

At step 118, in exemplary embodiments the setpoint relative humidity (RH) inside of the internal cavity for a moist heat decontamination cycle is in a range from about 20% RH to about 100% RH. In some preferred embodiments, the relative humidity inside of the internal cavity is in a range from about 50% RH to about 80% RH. In other embodiments, the relative humidity inside of the internal cavity is in a range from about 40% RH to about 100% RH, or even more particularly about 50% RH to about 100% RH, or even more particularly about 65% RH to about 90% RH. As such, the setpoint relative humidity for the decontamination cycle may be any value, range or subrange between or within the foregoing ranges, such as a RH of about any of 20%, 30%, 40%, 50%, 60%, 70%, 80%, 90%, or 100% (including any values or subranges between the stated values). The lower RH values may be particularly suitable for moisture sensitive objects, such as absorbent fabrics, for example. The lower RH values also are faster and easier to achieve, and may enable the unit to provide more repeatable results with a less complex structure of the decontamination unit. Increased RH values, however, may increase decontamination rate but may be at a cost of a more complex structure of the unit.

As shown at step 120, once the setpoint temperature and RH values have been achieved inside the internal cavity 14, the decontamination cycle is started. As such, the decontamination cycle and time thereof does not include the ramp up and ramp down times in the exemplary process. The objects to be contaminated may be placed in the internal cavity 14 during the ramp up, or may be placed in the cavity after the setpoints have been reached and before the start of the decontamination cycle.

As shown at steps 122 and 124, both the temperature and the humidity parameters (values) are maintained simultaneously during the decontamination cycle, which may include some degree of variation depending on the control of these parameters. Generally, the controller 20 uses information from the sensor(s) (e.g., temperature sensor 50 and humidity sensor 52, or humidity calculated by the same or separate temperature sensor 50) to maintain the temperature and humidity setpoints within the cavity 14 during the decontamination cycle. The controller 20 may use any suitable logic, such as PID-loop logic, to maintain the setpoint value inside of the internal cavity 14 within a certain error band, for example within 10% of the setpoint value.

The decontamination cycle is configured to run for a predetermined period of time according to the preset conditions, as shown at step 126. In exemplary embodiments, the decontamination cycle time is in a range from 15 minutes to 60 minutes, such as about any of 10, 15, 20, 25, 30, 35, 40, 45, 50, 55 or 60 minutes (including all values, ranges and subranges between the stated values). In exemplary embodiments, the decontamination cycle time is at both (i) the temperature value(s) within the temperature range (or temperature profile if used) and (ii) also is at the relative humidity value(s) within the relative humidity range (or humidity profile if used). It is understood, however, that either parameter of temperature or relative humidity within their respective ranges could run for only segment of the overall decontamination cycle time. By way of example and not limitation, at a decontamination cycle time of 15 minutes, the temperature could satisfy its value(s) in the above-noted range (e.g., 185° F.) for the full 15 minutes, whereas the relative humidity might satisfy its value(s) in its range (e.g., 65% RH) for only 10 minutes of the 15-minute cycle. In exemplary embodiments, however, it may be preferred to satisfy both parameters for the full cycle time, as shown in FIG. 10. Generally, shorter periods of time are particularly advantageous to increase throughput of decontaminating contaminated objects, which is especially beneficial in the event of a catastrophe, pandemic, or the like, where the availability of protective equipment may be limited and thus there may be a high desirability to decontaminate such equipment quickly. After the time has been satisfied, the decontamination cycle ends at step 128 and the object(s) are decontaminated. As noted above, in exemplary embodiments the decontamination cycle is performed without the use of chemicals, such that the exemplary unit is a chemical-free decontamination unit. A chemical-free decontamination unit and method provides advantages in terms of not having to handle or dispense of the chemicals, nor have separate devices to supply chemicals to the internal cavity 14. The use of chemicals, of course, may aid in the decontamination or sterilization of the object(s), and thus could be employed in some embodiments. For example, the chemical(s) could be added to the liquid in the humidity generator, or could be supplied by a separate device. Also as noted above, in exemplary embodiments the decontamination is performed at atmospheric pressure, which provides advantages in terms of reducing complexity of the unit 10, cycle time, etc. The use of positive pressure, of course, may provide advantages in terms of decontamination, and thus may be applied in some embodiments.

Decontamination, or disinfection, is quantified by inactivation rates or Log Reduction Value (or LRV). Log reduction is a simple mathematical term used to express the relative number of live microbes eliminated by disinfection. A 3 log reduction equates to 99.9%, a 4 log reduction equates to 99.99%, a 5 log reduction equates to 99.999%, a 6 log reduction equates to 99.9999%, a 7 log reduction equates to 99.99999%, etc.

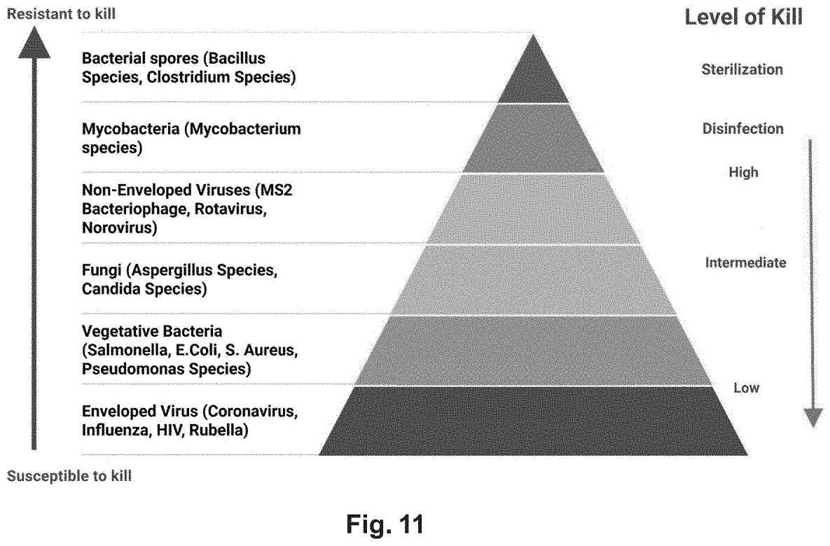

According to experimental data, detailed below, an exemplary decontamination cycle with a relatively fast cycle time (e.g., about 15 minutes), a relatively low temperature (e.g., about 185° F.), and a moderate relative humidity (e.g., about 65% RH) is found effective to provide at least between a 3 log and 7 log reduction in viral or bacterial loads including Mycobacteria and MS2 Bacteriophage. Based on at least these experimental results and other known kill rates of virus and bacteria (as shown in FIG. 11, for example), it is deduced that this exemplary decontamination cycle also would be effective to provide at least between a 3 log to 7 log reduction in other viral and/or bacterial loads, including but not limited to, enveloped virus (coronavirus, influenza, HIV, rubella), vegetative bacteria (salmonella, E. coli, S. aureus, pseudomonas species), fungi (aspergillus species, candida species), non-enveloped viruses (MS2 bacteriophage, rotavirus, norovirus), mycobacteria (Mycobacterium species), or the like. Furthermore, based on at least the experimental data detailed below, it is believed that the temperature and humidity values of the exemplary cycle could be expanded to include a temperature in a range from 170° F. to 200° F., a relative humidity in a range from 50% to 80%, and a cycle time in a range from 15 minutes to 60 minutes, while still being effective to provide at least a 3 log to 7 log reduction in the above-noted viral or bacterial loads, including enveloped virus, vegetative bacteria, fungi, non-enveloped viruses (MS2 bacteriophage, rotavirus, norovirus), mycobacteria (Mycobacterium species), or the like. These ranges of temperature, humidity, and time have been found to be repeatable and obtainable with a cost-effective unit that operates without a pressurized chamber and without expensive filtration.

The experimental data detailed below also demonstrates that increasing the temperature setpoint to about 212° F., increasing the relative humidity setpoint to about 100% RH, and increasing the cycle time about 60 minutes is effective to provide a greater than 6 log reduction (99.9999%) of difficult to kill spores (C. difficile spores). However, the test results indicate that the decontamination unit operating at atmospheric pressure demonstrated an inability to accurately measure and/or maintain relative humidity at temperatures of 100° C. This was especially true at the higher settings of relative humidity (i.e., 90% or 100% RH), which presents a difficulty to the operator since it becomes difficult to know when to place the mask inside the device, or whether the setpoint is being achieved.

Accordingly, in view of at least these experimental results, it is desirable to provide a cost-effective decontamination unit that provides significant (e.g., at least 3 log reduction) in viral and/or bacterial loads, such as enveloped virus, vegetative bacteria, fungi, non-enveloped viruses (MS2 bacteriophage, rotavirus, norovirus), mycobacteria (Mycobacterium species), or the like; but which specifically avoids more difficult to kill virus and/or bacteria such as endospores (e.g., Bacillus subtilis) or C. difficile spores, which otherwise require higher temperatures and/or humidity that is difficult to repeatably obtain with a cost-effective device.

### Experimental Data:

Three sets of exemplary (non-limiting) experiments were conducted as detailed below, in which each set of experiments used a moist heat decontamination unit as shown in FIGS. 7 and 8, and which included the following set of features:

Internals: The inside dimensions of the internal cavity is 555 mm wide, 685 mm deep, and 147 5 mm tall. Insulation is Fiberglass, thermal conductivity (K factor) is 0.23 at 75° F., 1½″ in doors, top, base; 2″ in sidewalls. Air tunnels include 22 ga. stainless steel; lift-out type, mounted on sides.

Base: The base is a one piece construction of 0.125 aluminum. A drip trough is provided formed of 18 ga. stainless steel mounted to front of cabinet with removable drip pan.

Hot unit components: Thermostat (holding): Solid state digital display control, room ambient to 200″F (93° C.). Thermostat (humidity): Solid state digital display control, room ambient to 95%. Switch: ON-OFF push button type. Power cord: Permanent, 10 fl., 12/3 ga. Heater: 2000 Watts for holding. Blower motor. Vent fan. Water pan: 4 gallons: 16 ga. stainless steel with 1850 Watt heater for humidity. Thermometer: Digital. Power: 2000 Watts, 120 Volts, 60 Hz., single phase, 16 Amps., 20 Amp service.

### First Set of Experiments:

In an exemplary (non-limiting) first set of experiments, the decontamination of MS2 bacteriophage was tested. This virus is a non-enveloped positive-stranded RNA virus of the bacteriophage family Leviviridae. Bacterial cells are the hosts for bacteriophages, and E. coli 15597 serves this purpose for MS2 bacteriophage. Its small size, icosahedral structure, and environmental resistance has made MS2 ideal for use as a surrogate virus (particularly in place of picornaviruses such as poliovirus and human norovirus) in water quality and disinfectant studies. The decontamination of the MS2 bacteriophage was tested according to the following procedure:

Summary of Procedure:
Test microorganism is prepared in appropriate liquid broth.
Test microorganism is harvested and the resulting suspension is diluted to achieve ≥1×106 CFU/mL.
Test and control carriers are inoculated and allowed to dry in optimal conditions for test microorganism.
Test carriers are placed in test device for the Sponsor-determined contact time.
Test carriers are harvested into liquid media and plated in optimal incubation conditions and time for the test microorganism.
After incubation, microbial concentrations are determined and reductions relative to pretreatment controls are calculated.

### Testing Parameters:

| | | | |
|---|---|---|---|
| Host Culture | Tryptic | Culture | 6-18 hours |
| Growth | Soy Broth | Growth Time | |
| Media | | | |
| Culture | Phosphate | Culture | N/A |
| Dilution | Buffered | Supplement | |
| Media | Saline | | |
| Carrier Type | See Notes | Inoculum | 0.030 ml |
| | | Volume | |
| Carrier Dry | 20-40 minutes | Carrier Dry | Ambient |
| Type | | Temp. and | |
| | | Humidity | |
| Contact Time | 15 minutes | Contact | 185° F./65% RH |
| | | Temperature/ | |
| | | Humidity | |
| Harvest | Phosphate | Enumeration | 50% Tryptic |
| Media | Buffered | Media | Soy Broth |
| (Volume) | Saline with | | |
| | 0.1 % Tween- | | |
| | 80 (20 ml) | | |
| Incubation | 36° C. | Incubation | 12-24 hours |
| Temperature | | Time | |

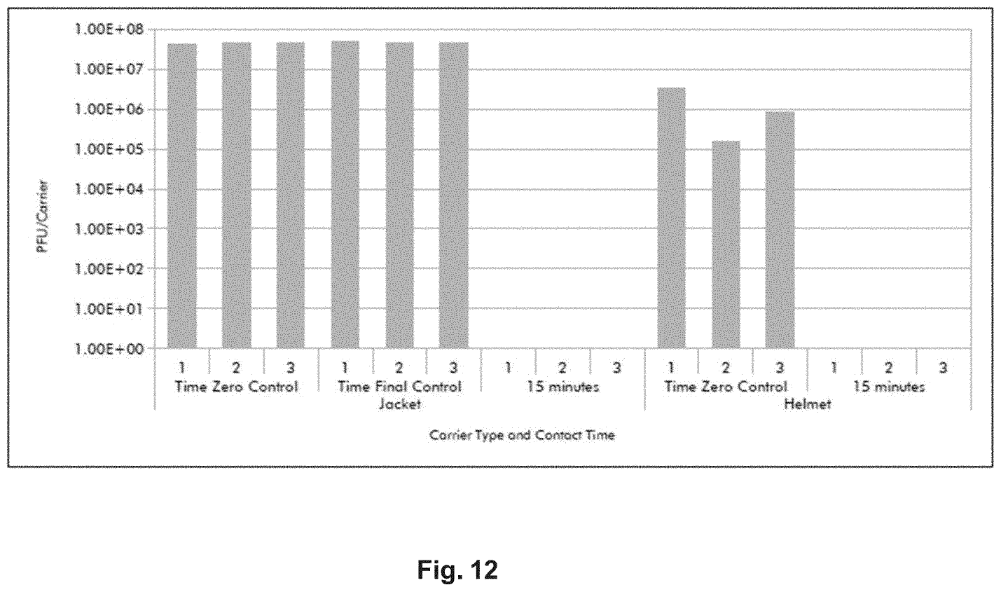

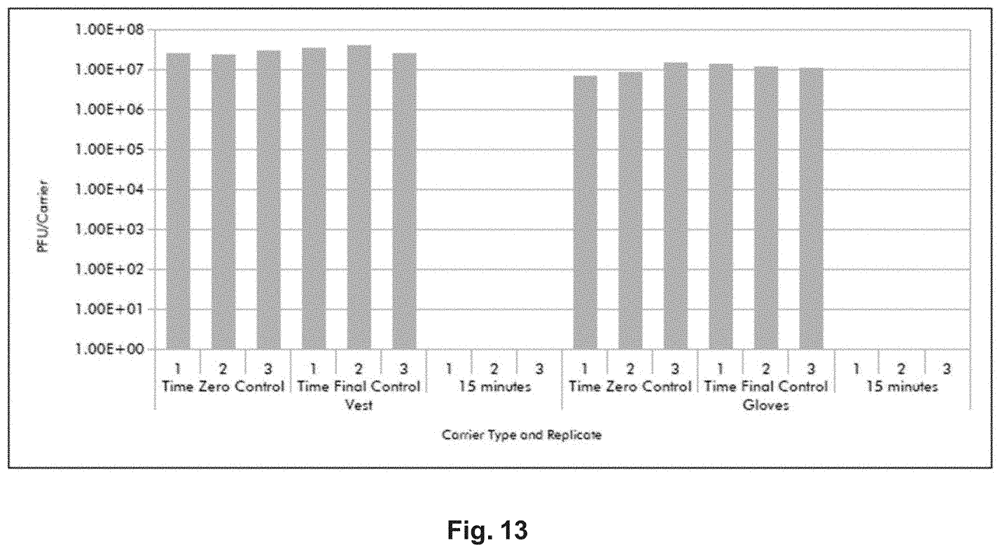

The carriers for this study were: Firefighter Turnout Jacket, Gloves, Helmet and Ballistic Vest. For each set of parameters and microorganisms evaluated, all carriers were treated simultaneously within the test device. The jacket, gloves and vest control and test carriers were all harvested by cutting out the inoculated section of the carrier into 20 mL of harvest media. The jacket and vest were inoculated on the chest portion of the carriers, while the gloves were inoculated on both sides of the fingertips. As each inoculation site was removed for harvesting, the locations were slightly different between each run. The helmet control and test carriers were harvested via sterile swabbing of the inoculation site. No time final controls were able to be taken for the helmet due to test method constraints. In the results section, the reductions for the helmet are calculated based on the time zero controls. Time zero and time final controls were taken for all other test carriers. After loading the inoculated carriers into the test device, the contact time was not started until the temperature and humidity values reached their target values. Carriers were loaded as quickly as possible to reduce the amount of time the device needed to reach the prescribed parameters. Results of the study are included in Table 1 and also are shown in FIGS. 12 and 13.

TABLE 1
Results of the Study (185° F./65% RH/15 minutes)

| | | | | | | Percent | Log |
|---|---|---|---|---|---|---|---|
| | | | | | | Reduction | Reduction |
| | | | | | | Compared | Compared |
| | | | | | Average | to Control | to Control |
| Test | Test | Contact | | PFU/ | PFU/ | at Time | at Time |
| Microorganism | Article | Time | Replication | Carrier | Carrier | Zero | Zero |
| MS2 | Jacket | Time | 1 | 4.20E+07 | 4.32E+07 | N/A | |
| Bacteriophage | | Zero | 2 | 4.40E+07 | | | |
| ATCC | | Control | 3 | 4.36E+07 | | | |
| 15597-B1 | | Time | 1 | 5.00E+07 | 4.73E+07 | No Reduction | |
| | | Final | 2 | 4.56E+07 | | | |
| | | Control | 3 | 4.64E+07 | | | |
| | | 15 | 1 | <1.00E+01 | <1.00E+01 | >99.99998% | >6.64 |
| | | Minutes | 2 | <1.00E+01 | | | |
| | | | 3 | <1.00E+01 | | | |
| | Helmet | Time | 1 | 3.40E+06 | 1.45E+06 | N/A | |
| | | Zero | 2 | 1.5E+05 | | | |
| | | Control | 3 | 8.00E+05 | | | |
| | | Time | N/A | | | | |
| | | Final | | | | | |
| | | Control | | | | | |
| | | 15 | 1 | <1.00E+01 | <1.00E+01 | >99.9993% | >5.16 |
| | | Minutes | 2 | <1.00E+01 | | | |
| | | | 3 | <1.00E+01 | | | |
| | Vest | Time | 1 | 2.52E+07 | 2.54E+07 | N/A | |
| | | Zero | 2 | 2.23E+07 | | | |
| | | Control | 3 | 2.87E+07 | | | |
| | | Time | 1 | 3.42E+07 | 3.29E+07 | No Reduction | |
| | | Final | 2 | 3.92E+07 | | | |
| | | Control | 3 | 2.54E+07 | | | |
| | | 15 | 1 | <1.00E+01 | <1.00E+01 | >99.99996 | >6.40 |
| | | Minutes | 2 | <1.00E+01 | | | |
| | | | 3 | <1.00E+01 | | | |
| | Gloves | Time | 1 | 6.70E+06 | 9.67+06 | N/A | |
| | | Zero | 2 | 8.20E+06 | | | |
| | | Control | 3 | 1.41E+07 | | | |
| | | Time | 1 | 1.31E+07 | 1.21E+07 | No Reduction | |
| | | Final | 2 | 1.21E+07 | | | |
| | | Control | 3 | 1.20E+07 | | | |
| | | 15 | 1 | <1.00E+01 | <1.00E+01 | >99.999897% | >5.99 |
| | | Minutes | 2 | <1.00E+01 | | | |
| | | | 3 | <1.00E+01 | | | |

Note:
The lower limit of detection for this study was 1.00E+01 PFR/mL. Values observed less than the limit are reported as “<1.00E+01” in results table and zero in the graph.

Based on the results, it is found that humidified heat within an enclosed system can be used for high-level decontamination of first responder equipment.

Specifically, it is found that MS2 bacteriophage decontamination is rapidly and easily achieved at settings as low as 85° C. and 65% relative humidity for 15 minutes duration.

### Second Set of Experiments:

In an exemplary (non-limiting) first set of experiments, the decontamination of Bacillus subtilis endospores was tested. This is a difficult to kill bacteria which is Gram-positive, rod shaped, capable of forming endospores. Endospores of Bacillus subtilis can tolerate harsh environmental conditions such as UV exposure and high temperatures. Typically found in soil, this species is not known to cause disease in healthy individuals, but can be considered an opportunistic pathogen among the immuno-compromised. Bacillus subtilis endospores serve as one of the models for evaluating the effectiveness of sporicides and sterilants. The decontamination of Bacillus subtilis was tested according to the following procedure:

Summary of the Procedure:
Test microorganism is prepared in appropriate liquid broth.
Test microorganism is harvested and the resulting suspension is diluted to achieve ≥1×106 CFU/mL.
Test and control carriers are inoculated and allowed to dry in optimal conditions for test microorganism.
Test carriers are placed in test device for the Sponsor-determined contact time.
Test carriers are harvested into liquid media and plated in optimal incubation conditions and time for the test microorganism.
After incubation, microbial concentrations are determined and reductions relative to pretreatment controls are calculated.

Testing Parameters:

| | | | |
|---|---|---|---|
| Culture | N/A-Fride | Culture | N/A-Fride Stock |
| Growth | Stock | Growth | |
| Media | | Time | |
| Culture | Phosphate | Culture | with 0.1% |
| Dilution | Buffered | Supplement | Tween-80 |
| Media | Saline | | (20 ml) |
| Carrier | See Notes | Inoculum | 0.030 ml |
| Type | | Volume | |
| Carrier Dry | 20-40 | Carrier | Ambient |
| Type | minutes | Dry Temp. | |
| | | and Humidity | |
| Contact | 15 and 60 | Contact | 185° F./65% RH (Rd. 1) |
| Time | minutes | Temperature/ | 200° F./65% RH (Rd. 2) |
| | | Humidity | 200° F./90% RH (Rd. 3) |
| Harvest | Phosphate | Enumeration | Tryptic Soy Broth |
| Media | Buffered | Media | |
| (Volume) | Saline | | |
| | with 0.1% | | |
| | Tween-80 | | |
| | (20 ml) | | |
| Incubation | 36° C. | Incubation | 24-28 hours |
| Temperature | | Time | |

The carriers for this study were: Firefighter Turnout Jacket, Gloves, Helmet and Ballistic Vest. For each set of parameters and microorganisms evaluated, all carriers were treated simultaneously within the test device. The jacket, gloves and vest control and test carriers were all harvested by cutting out the inoculated section of the carrier into 20 mL of harvest media. The jacket and vest were inoculated on the chest portion of the carriers, while the gloves were inoculated on both sides of the fingertips. As each inoculation site was removed for harvesting, the locations were slightly different between each run. The helmet control and test carriers were harvested via sterile swabbing of the inoculation site. No time final controls were able to be taken for the helmet due to test method constraints. In the results section, the reductions for the helmet are calculated based on the time zero controls. Time zero and time final controls were taken for all other test carriers. Due to limited fabric of the test carriers and because the inoculum was identical for each set of parameters evaluated, the time zero and time final controls were shared between the second and third rounds of B. subtilis testing. After loading the inoculated carriers into the test device, the contact time was not started until the temperature and humidity values reached their target values. Carriers were loaded as quickly as possible to reduce the amount of time the device needed to reach the prescribed parameters.

During the third round of B. subtilis testing, it was noted that at the 90% RH setting the device was leaking water. Extra water was added when loading the test carriers and the humidity setting was lowered to 85% RH. No leakage was observed during the contact time.

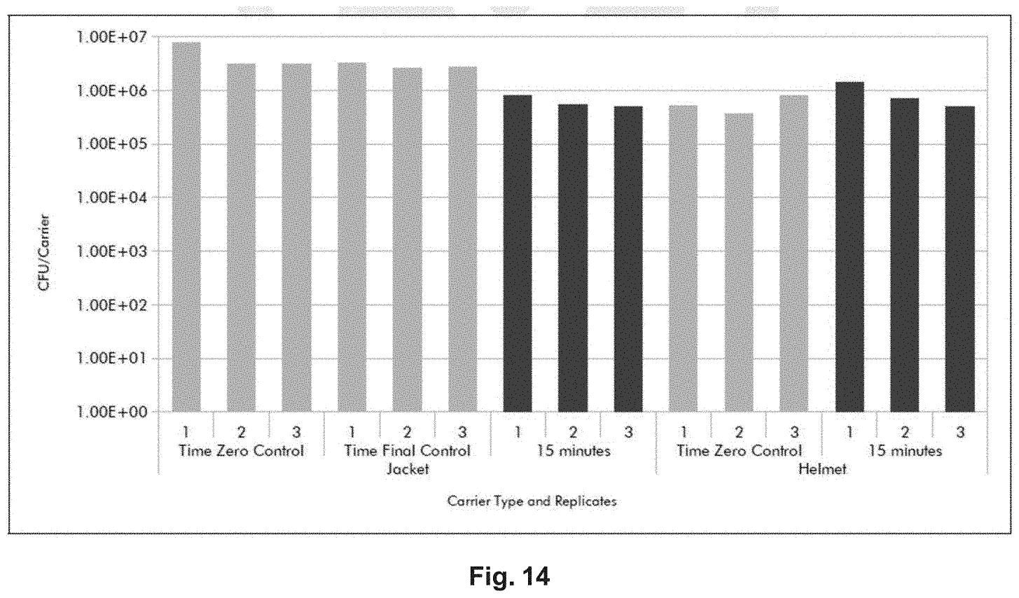

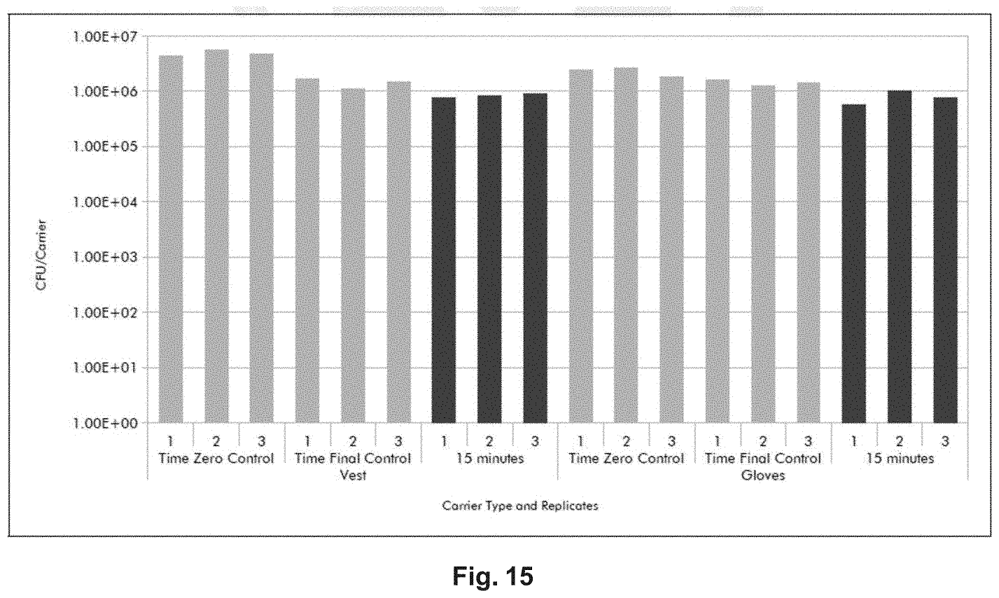

Results of the study are included in Tables 2-4 and also are shown in FIGS. 14-19.

TABLE 2
Results of the Study (Round 1, 185° F./65% RH/15 minutes)

| | | | | | | Percent | Log |
|---|---|---|---|---|---|---|---|
| | | | | | | Reduction | Reduction |
| | | | | | | Compared | Compared |
| | | | | | Average | to Control | to Control |
| Test | Test | Contact | | PFU/ | PFU/ | at Time | at Time |
| Microorganism | Article | Time | Replication | Carrier | Carrier | Zero | Zero |
| Bacillus subtilis | Jacket | Time | 1 | 7.80E+06 | 4.67E+06 | N/A | |
| ATCC 19659 | | Zero | 2 | 3.10E+06 | | | |
| (endoscopes) | | Control | 3 | 3.10E+06 | | | |
| | | Time | 1 | 3.20E+06 | 2.83E+06 | 39.29% | 0.22 |
| | | Final | 2 | 2.60E+06 | | | |
| | | Control | 3 | 2.70E+06 | | | |
| | | 15 | 1 | 7.92E+05 | 6.11E+05 | 86.90% | 0.88 |
| | | Minutes | 2 | 5.32E+05 | | | |
| | | | 3 | 5.10E+05 | | | |
| | Helmet | Time | 1 | 5.30E+05 | 5.73E+05 | 87.71% | 0.91 |
| | | Zero | 2 | 3.70E+05 | | | |
| | | Control | 3 | 8.30E+05 | | | |
| | | Time | N/A | | | | |
| | | Final | | | | | |
| | | Control | | | | | |
| | | 15 | 1 | 1.40+06 | 8.67E+05 | No Reduction | |
| | | Minutes | 2 | 7.00E+−5 | | | |
| | | | 3 | <1.00E+01 | | | |
| Bacillus subtilis | Vest | Time | 1 | 4.30E+06 | 4.83E+06 | N/A | |
| ATCC 19659 | | Zero | 2 | 5.50E+06 | | | |
| (endoscopes) | | Control | 3 | 4.70E+06 | | | |
| | | Time | 1 | 1.65E+06 | 1.40E+06 | 70.97% | 0.54 |
| | | Final | 2 | 1.09E+06 | | | |
| | | Control | 3 | 1.47E+06 | | | |
| | | 15 | 1 | 7.56E+0S | 8.33E+05 | 82.76% | 0.76 |
| | | Minutes | 2 | 8.36E+0S | | | |
| | | | 3 | 9.08E+05 | | | |
| | Gloves | Time | 1 | 2.37E+06 | 2.28E+06 | N/A | |
| | | Zero | 2 | 2.67E+06 | | | |
| | | Control | 3 | 1.79E+06 | | | |
| | | Time | 1 | 1.61E+06 | 1.42E+06 | 37.78% | 0.21 |
| | | Final | 2 | 1.23E+06 | | | |
| | | Control | 3 | 1.41E+06 | | | |
| | | 15 | 1 | 5.78E+0S | 7.77E+05 | 65.86% | 0.47 |
| | | Minutes | 2 | 1.00E+06 | | | |
| | | | 3 | 7.54E+0S | | | |

The data in Table 2 corresponds with the data shown in FIGS. 14 and 15.

TABLE 3
Results of the Study (Round 2, 200° F./65% RH/60 minutes

| | | | | | | Percent | Log |
|---|---|---|---|---|---|---|---|
| | | | | | | Reduction | Reduction |
| | | | | | | Compared | Compared |
| | | | | | Average | to Control | to Control |
| Test | Test | Contact | | PFU/ | PFU/ | at Time | at Time |
| Microorganism | Article | Time | Replication | Carrier | Carrier | Zero | Zero |
| Bacillus subtilis | Jacket | Time | 1 | 7.80E+06 | 4.67E+06 | N/A | |
| ATCC 19659 | | Zero | 2 | 3.10E+06 | | | |
| (endoscopes) | | Control | 3 | 3.10E+06 | | | |
| | | Time | 1 | 2.80E+06 | 2.3E+06 | 50.00% | 0.3 |
| | | Final | 2 | 2.00E+06 | | | |
| | | Control | 3 | 2.20E+06 | | | |
| | | 15 | 1 | 1.20E+04 | 3.00E+06 | 98.71% | 1.89 |
| | | Minutes | 2 | 6.00E+03 | | | |
| | | | 3 | 7.20E+04 | | | |
| | Helmet | Time | 1 | 5.30E+05 | 5.73E+05 | 87.71% | 0.91 |
| | | Zero | 2 | 3.70E+05 | | | |
| | | Control | 3 | 8.20E+05 | | | |
| | | Time | N/A | | | | |
| | | Final | | | | | |
| | | Control | | | | | |
| | | 15 | 1 | 3.00E+01 | 1.09E+03 | 99.81% | 2.72 |
| | | Minutes | 2 | 4.00E+01 | | | |
| | | | 3 | 3.19E+03 | | | |
| Bacillus subtilis | Jacket | Time | 1 | 7.80E+06 | 4.67E+06 | N/A | |
| ATCC 19659 | | Zero | 2 | 3.10E+06 | | | |
| (endoscopes) | | Control | 3 | 3.10E+06 | | | |
| | | Time | 1 | 2.80E+06 | 2.33E+06 | 50.00% | 0.3 |
| | | Final | 2 | 2.00E+06 | | | |
| | | Control | 3 | 2.20E+06 | | | |
| | | 15 | 1 | 1.20E+04 | 3.00E+04 | 98.71% | 1.89 |
| | | Minutes | 2 | 6.00E+03 | | | |
| | | | 3 | 7.20E+04 | | | |
| | Helmet | Time | 1 | 5.30E+05 | 5.73E+05 | 87.71% | 0.91 |
| | | Zero | 2 | 3.70E+05 | | | |
| | | Control | 3 | 8.20E+05 | | | |
| | | Time | N/A | | | | |
| | | Final | | | | | |
| | | Control | | | | | |
| | | 15 | 1 | 3.00E+01 | 1.09E+03 | 99.81% | 2.72 |
| | | Minutes | 2 | 4.00E+01 | | | |
| | | | 3 | 3.19E+03 | | | |
| Bacillus subtilis | Vest | Time | 1 | 4.30E+06 | 4.83E+06 | N/A | |
| ATCC 19659 | | Zero | 2 | 5.50E+06 | | | |
| (endoscopes) | | Control | 3 | 4.70E+06 | | | |
| | | Time | 1 | 8.00E+05 | 7.07E+05 | 85.38% | 0.84 |
| | | Final | 2 | 6.50E+05 | | | |
| | | Control | 3 | 6.70E+0S | | | |
| | | 15 | 1 | 5.40E+04 | 3.63E+04 | 99.25% | 2.12 |
| | | Minutes | 2 | 2.40E+04 | | | |
| | | | 3 | 3.10E+04 | | | |
| | Gloves | Time | 1 | 2.37E+06 | 2.28E+06 | N/A | |
| | | Zero | 2 | 2.67E+06 | | | |
| | | Control | 3 | 1.79E+06 | | | |
| | | Time | 1 | 2.30E+06 | 2.40E+06 | No Reduction | |
| | | Final | 2 | 2.40E+06 | | | |
| | | Control | 3 | 2.50E+06 | | | |
| | | 15 | 1 | 5.00E+03 | 7.89E+04 | 96.53% | 1.46 |
| | | Minutes | 2 | 4.76E+03 | | | |
| | | | 3 | 2.27E+05 | | | |

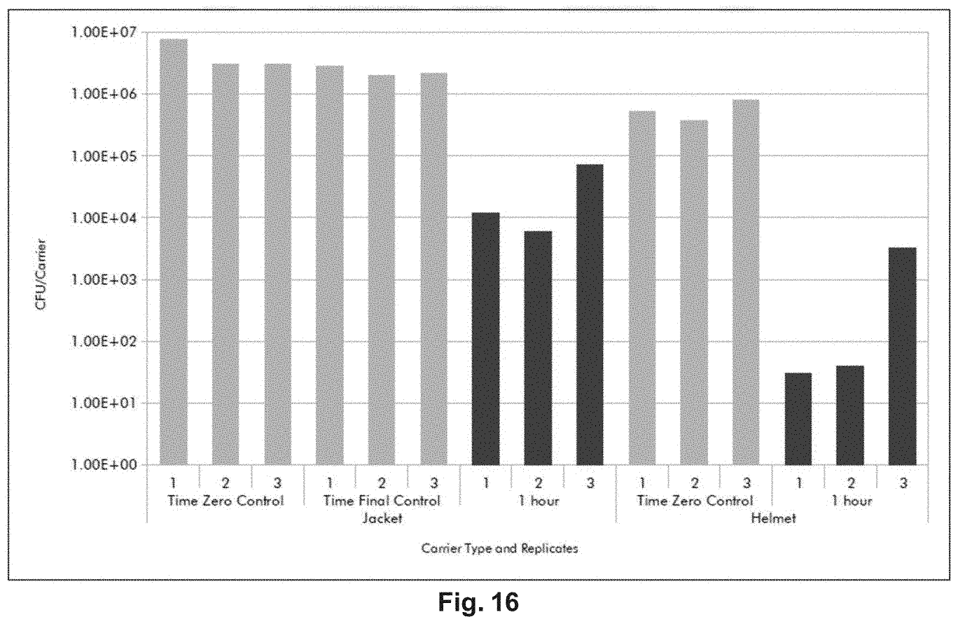

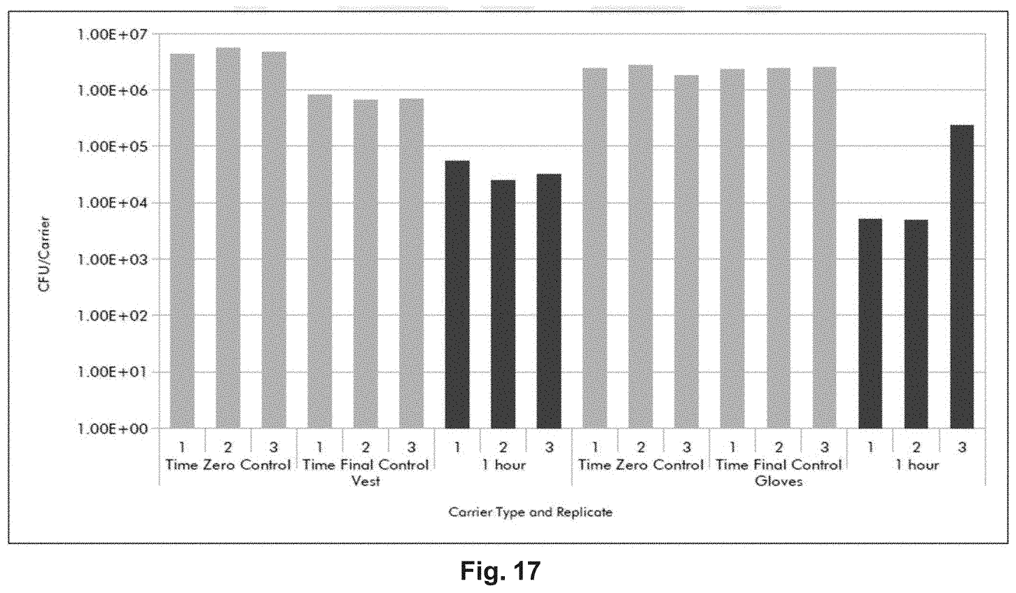

The data in Table 3 corresponds with the data shown in FIGS. 16 and 17.

TABLE 4
Results of the Study (Round 3, 200° F./90% RH/60 minutes)

| | | | | | | Percent | Log |
|---|---|---|---|---|---|---|---|
| | | | | | | Reduction | Reduction |
| | | | | | | Compared | Compared |
| | | | | | Average | to Control | to Control |
| Test | Test | Contact | | PFU/ | PFU/ | at Time | at Time |
| Microorganism | Article | Time | Replication | Carrier | Carrier | Zero | Zero |
| Bacillus subtilis | Jacket | Time | 1 | 7.80E+06 | 4.67E+06 | N/A | |
| ATCC 19659 | | Zero | 2 | 3.10E+06 | | | |
| (endoscopes) | | Control | 3 | 3.10E+06 | | | |
| | | Time | 1 | 2.80E+06 | 2.33E+06 | 50.00% | 0.30 |
| | | Final | 2 | 2.00E+06 | | | |
| | | Control | 3 | 2.20E+06 | | | |
| | | 15 | 1 | 2.20E+06 | 2.10E+06 | 10.00% | 0.05 |
| | | Minutes | 2 | 1.70E+06 | | | |
| | | | 3 | 2.40E+06 | | | |
| | | Time | 1 | 5.30E+06 | 5.73E+05 | 97.71% | 0.91 |
| | | Zero | 2 | 3.70E+05 | | | |
| | | Control | 3 | 8.20E+07 | | | |
| | | Time | 1 | N/A | | | |
| | Helmet | Final | 2 | | | | |
| | | Control | 3 | | | | |
| | | 15 | 1 | 4.71E+05 | 7.77E+05 | No Reduction | |
| | | Minutes | 2 | 4.60E+0S | | | |
| | | | 3 | 1.40E+06 | | | |
| Bacillus subtilis | | Time | 1 | 4.30E+06 | 4.83E+06 | N/A | |
| ATCC 19659 | | Zero | 2 | 5.50E+06 | | | |
| (endoscopes) | | Control | 3 | 4.70E+06 | | | |
| | | Time | 1 | 8.00E+05 | 7.07E+05 | 85.38% | 0.84 |
| | | Final | 2 | 6.50E+05 | | | |
| | | Control | 3 | 6.70E+05 | | | |
| | | 15 | 1 | 4.15E+05 | 8.70E+05 | 82.01% | 0.74 |
| | | Minutes | 2 | 1.80E+06 | | | |
| | | | 3 | 3.94E+05 | | | |
| | | Time | 1 | 2.37E+06 | 2,28E+06 | N/A | |
| | | Zero | 2 | 2.67E+06 | | | |
| | | Control | 3 | 1.79E+06 | | | |
| | | Time | 1 | 2.30E+06 | 2.40E+06 | No Reduction | |
| | | Final | 2 | 2.40E+06 | | | |
| | | Control | 3 | 2.50E+06 | | | |
| | | 15 | 1 | 4.08E+05 | 3.93E+05 | 82.68% | 0.76% |
| | | Minutes | 2 | 4.00E+05 | | | |
| | | | 3 | 3.75E+05 | | | |

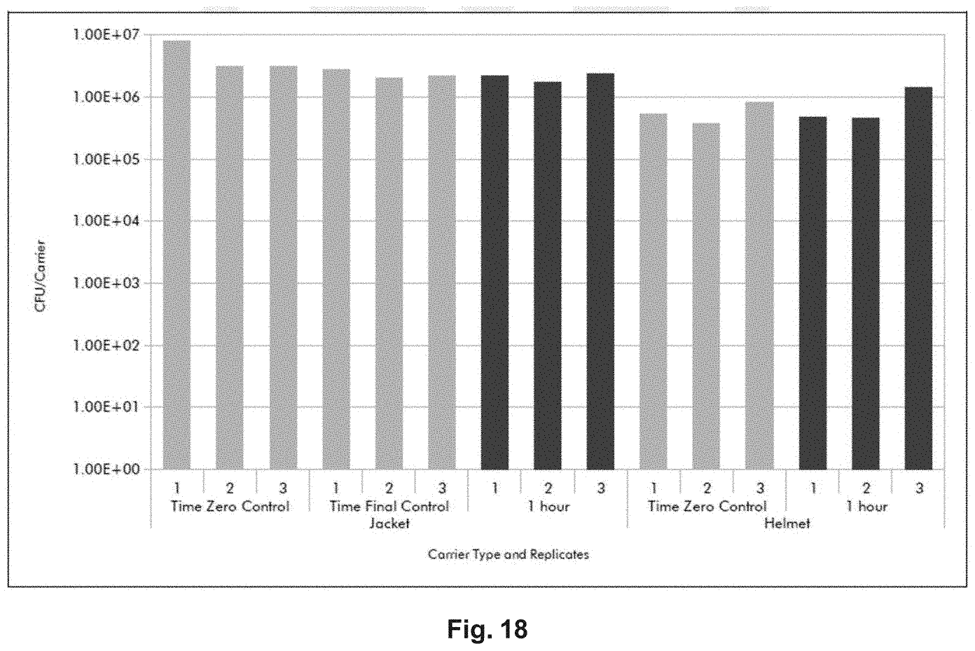

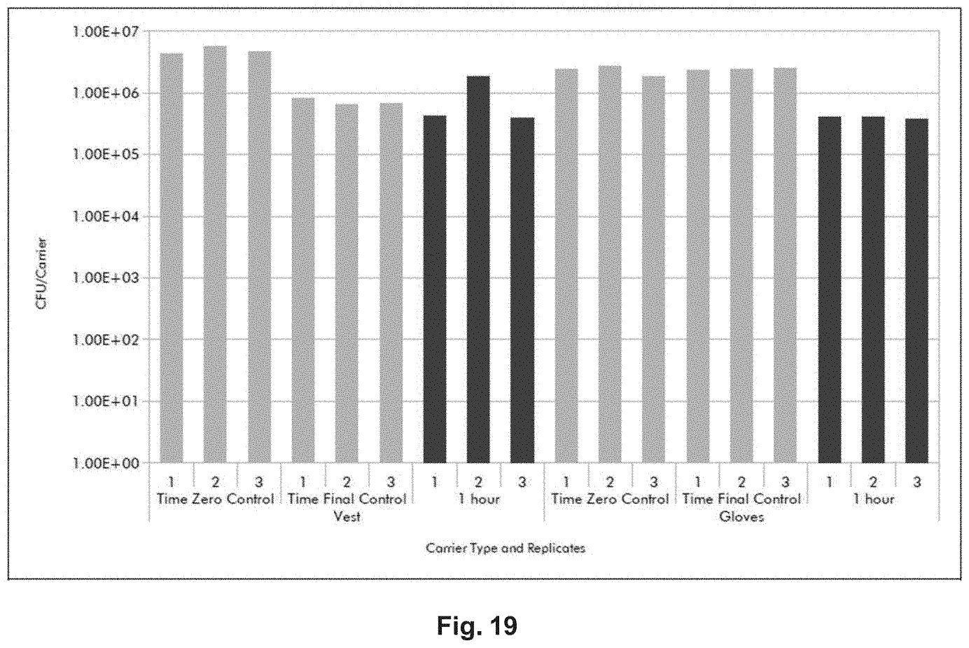

The data in Table 4 corresponds with the data shown in FIGS. 18 and 19.

Based on the results of the second set of experiments, it is found that Bacillus subtilis is difficult to effectively reduce with the decontamination unit, especially at higher humidity levels where the setpoint of 90% RH was difficult to achieve during testing. It also is shown between Rounds 2 and 3 of the testing that the 90% humidity setpoint was not as effective at reducing the Bacillus subtilis (endospores) as was the 65% humidity setpoint.

### Third Set of Experiments:

In an exemplary (non-limiting) third set of experiments, the decontamination of MS2 bacteriophage, mycobacteria abscessus, and C. difficile spores was tested according to the following procedure:

The model 1860 N95 (3M, Minneapolis, USA) respirator was studied. The test and control respirators were inoculated with ˜10⁶ colony-forming units (CFU) of Clostridium difficile spores, ˜10⁶ colony-forming units (CFU) of Mycobacterium abscessus and ˜10⁶ plaque-forming units (PFU) of bacteriophage MS2 on the outer and inner surface of the respirator. The test organisms were suspended in 8% simulated mucus, and 10 μL aliquots were pipetted onto the respirator surface and spread with a sterile loop to cover an area of 1 cm² and allowed to air dry. The test N95 respirators were suspended using metallic CS' shaped hooks from metal wire shelving carts at the topmost shelf within the cabinet and subjected to humidified heat. The control masks were left untreated at room temperature.

After the disinfection treatments, the inoculated sections of the N95 respirators were cut out, vortexed for 1 minute in 1 mL of phosphate-buffered saline with 0.02% Tween and serial dilutions are plated on selective media to quantify viable organisms. Broth enrichment cultures are used to assess for recovery of low levels of C. difficile spores. All tests are performed in triplicate. Log₁₀ CFU or PFU reductions are calculated by comparing recovery from treated versus untreated control respirators.

Disinfection with moist heat is conducted within decontamination unit described above. The N95 FFRs being decontaminated are hanged within the modified shelfing at regular intervals. Tests are performed at settings ranging from temperatures of 85° C.-100° C., a relative humidity setting of 65%-100% and time of 15 minutes to 60 minutes. The masks are inserted into the cabinet after reaching the setpoint temperature and relative humidity values. During the insertion, opening of the cabinet door results in brief drop in the temperature and relative humidity setpoint values. No chemicals are added to the decontamination process. The decontamination cycle is performed at ambient pressure.

The results of testing demonstrate that both relative humidity (RH) and duration of exposure affected the disinfection capabilities of humidified heat. At 85° C. and 65% RH for 15 minutes there is >6 log reduction of MS2 bacteriophage and Mycobacterium abscessus. 6 log reduction of C. difficile spores is achieved after 1 hour of exposure at 100° C. and at a setting of 100% RH on the device. The results are as follows in Table 1.

TABLE 1
Test Results

| | | Relative | Duration | Log |
|---|---|---|---|---|
| | | Humidity | of | reduction |
| | Temperature | setting on | Exposure | (PFU/ |
| Test Organism | (° C.) | device (%) | (minutes) | CFU) |
| MS2 | 85 | 65 | 15 | 6.151 |
| Bacteriophage | 85 | 85 | 60 | 7 |
| Mycobacterium | 85 | 65 | 15 | 7.111 |
| abscessus | 85 | 65 | 30 | 7.111 |
| | 85 | 65 | 60 | 7.111 |
| C. Difficile | 85 | 65 | 30 | 1.824 |
| Spores | 85 | 85 | 30 | 2.495 |
| | 85 | 85 | 60 | 3.23 |
| | 85 | 100 | 60 | 3.179 |
| | 100 | 100 | 60 | 6 |
| | 100 | 75 | 60 | 5.486 |
| | 100 | 65 | 60 | 4.104 |

A summary of the results in Table 1 is provided in Table 2, below. Based on the results, it is found that humidified heat within an enclosed system can be used for high-level decontamination of N95 FFRs.

Specifically, it is surprisingly found that viral and mycobacterial decontamination is rapidly and easily achieved at settings as low as 85° C. (185° F.) and 65% relative humidity for 15 minutes duration.

TABLE 2
Summary of Results

| CYCLE | EFFICIENCY |
|---|---|
| 185 F., 65% | >6 log reduction of MS2 Bacteriophage |
| Humidity setting, | (non-lipid surrogate for Sars-Cov-2) |
| 15 min cycle | >6 log reduction of Mycobacteria |
| 212 F., 100% | >6 log reduction of difficult |
| Humidity setting, | to kill spores |
| 60 min cycle | (C. difficile spores) |

Moreover, it is found that greater than 6 log spore reduction can be achieved at higher settings of 100° C. and 100% relative humidity on the device for 1 hour duration. However, the test results indicate that the decontamination unit operating at atmospheric pressure demonstrated an inability to accurately measure relative humidity at temperatures of 100° C. This was especially true at the higher settings of relative humidity (i.e., 100% RH), which presents a difficulty to the operator since it becomes difficult to know when to place the mask inside the device. At a temperature of 100° C. and setting of 100%, the highest actual RH noted was 77% that the decontamination unit achieved.

In view of at least the foregoing three experimental sets of testing, the decontamination unit may be specifically adapted to be cost-effective and robust enough to provide a repeatable time, temperature, and humidity cycle that can significantly reduce certain bacterial and viral load(s), but the unit is not so complex as to significantly reduce other more difficult to eliminate viruses and bacteria. For example, based on at least the experimental data, it is deduced that a decontamination cycle including a temperature in a range from 170° F. to 200° F., a relative humidity in a range from 50% to 80%, and a cycle time in a range from 15 minutes to 60 minutes is effective to provide at least a 3 log to 7 log reduction in viral or bacterial loads, including but not limited to, enveloped virus (coronavirus, influenza, HIV, rubella), vegetative bacteria (salmonella, E. coli, S. aureus, pseudomonas species), fungi (aspergillus species, candida species), non-enveloped viruses (MS2 bacteriophage, rotavirus, norovirus), and mycobacteria (Mycobacterium species), or equivalents. However, other bacteria and virus such as bacterial spores (Bacillus species (Bacillus subtilis), Clostridium species, C. difficile spores), or equivalents are not intended to be significantly reduced (e.g., less than 3 log reduction) by the exemplary decontamination unit, as this may otherwise require relatively high temperature and humidity (or excessive pressure/vacuum) that is difficult to maintain, repeatedly provide, or otherwise may demand a greater complexity that is not a cost-effective solution, such as for everyday use at first responder locations, schools, or the like. In view of the data,

According to another aspect, a method of decontaminating first responder equipment includes: (i) placing the first responder equipment within an internal cavity of a decontamination unit; supplying heat to the internal cavity; supplying humidity to the internal cavity for a time; wherein the supplying heat and the supplying humidity is effective to provide between a 3 log to 7 log reduction in bacterial or viral load. The method may be effective to provide such a reduction in enveloped virus, vegetative bacteria, fungi, non-enveloped viruses, mycobacteria (Mycobacterium species), or the like; but may be ineffective at reducing viral or bacterial spores (Bacillus species (Bacillus subtilis), Clostridium species, C. difficile spores, or the like.

An exemplary moist heat decontamination unit and method has been described herein. The decontamination unit includes a housing having an internal cavity for containing a contaminated object that is to be decontaminated, a heater in thermal communication with the internal cavity and is configured to heat the internal cavity and object, and a humidity generator in fluid communication with the internal cavity and configured to supply humidity to the internal cavity. A controller is configured to control a temperature and a relative humidity within the internal cavity according to a decontamination cycle for decontaminating the object. The decontamination cycle may be preset based on the object(s) and/or type of contaminant.

According to one aspect, the decontamination cycle is performed with at least the parameters of a temperature in a range from 140° F. to 220° F., a relative humidity in a range from 50% to 100%, and a time in a range from 15 minutes to 60 minutes. In exemplary embodiments, the decontamination cycle may be performed without the use of chemicals. In exemplary embodiments, the decontamination cycle may be performed at atmospheric pressure.

According to a preferred aspect, the decontamination cycle is performed with at least the parameters of a temperature in a range from 170° F. to 200° F., a relative humidity in a range from 50% to 80%, and a time in a range from 15 minutes to 60 minutes.

As is apparent from the foregoing description, the exemplary moist heat decontamination unit and method includes many advantages, including one or more advantages of: (i) a relatively low-temperature decontamination cycle with an associated relative humidity for decontaminating an object; (ii) decontamination at atmospheric pressure; (iii) decontamination without the use of chemicals; (iv) a relatively short cycle time for increasing decontamination throughput of contaminated objects, which may be particularly advantageous in the event of a catastrophe or pandemic, for example; (v) a relatively inexpensive unit that is affordable and deployable at various points of use, such as at schools, police stations, fire stations, department of defense, other government agencies, elderly care facilities, hospitals, and the like; and/or (vi) an enhanced modularity or tailorability of the internal object-support or racking system design for enabling decontamination of many different types of objects, such as first responder service wear (e.g., paramedic or firefighter protective gear), or the like.

Specifically, research by the inventors indicates that significantly no appropriate localized solution has been found to exist for decontamination of service wear, protective gear, PPE, etc., beyond merely wiping down and spraying occasionally.

The exemplary decontamination unit and method described herein may thus provide improvements including one or more of: using a chemical-free method that could be deployed at the point of use, which requires almost no training; saves worker time and removes the variability between users; saves space—i.e., takes up very little floor space (3′×3′); designed for mobility around multiple sites; runs on standard 110V (20/15 Amp) and can be made in 240V configuration to support overseas operation; offers localized decontamination which reduces waiting time, prevents infection and cross-contamination; demonstrates >6 log reduction (99.9999%) upon testing on resistant virus, bacteria, and/or spores (with higher resistance than SARS-COV-2); uses no chemicals or gases, hence no degassing and no residual toxicity; can be used to decontaminate items that can withstand heat and humidity such as jackets, vests, gloves, boots, goggles, helmets, turnout gear, etc.

Generally, the exemplary decontamination unit provides one or more advantages of: being easily replicable and can be scaled up; short cycle times which is advantageous in times of need; use of just water as the disinfectant agent, where concerns for supply chain disruptions would not be an issue; relatively low temperatures where there is little or no concern for off-gassing from masks and/or other objects to be decontaminated.

According to an aspect, a decontamination unit includes: a housing having an internal cavity and an opening for accessing the internal cavity; a door coupled to the housing for opening or closing the opening for permitting or restricting access to the internal cavity; a heater in thermal communication with the internal cavity and configured to heat the internal cavity; a humidity generator in fluid communication with the internal cavity and configured to supply humidity to the internal cavity; and a controller configured to control a temperature and a relative humidity within the internal cavity according to a preset decontamination cycle; wherein the preset decontamination cycle is configured to sufficiently decontaminate an object based on the object to be decontaminated and the contaminant on the object.

According to another aspect, a decontamination unit includes: a housing having an internal cavity and an opening for accessing the internal cavity; a door coupled to the housing for opening or closing the opening for permitting or restricting access to the internal cavity; a heater in thermal communication with the internal cavity and configured to heat the internal cavity; a humidity generator in fluid communication with the internal cavity and configured to supply humidity to the internal cavity; a controller operatively coupled to at least the heater, the controller being configured to control a temperature and a relative humidity within the internal cavity according to a preset decontamination cycle having at least the following parameters: the temperature of the preset decontamination cycle is in a range from 170° F. to 200° F.; the relative humidity of the preset decontamination cycle is in a range from 50% to 80%; and a time of the preset decontamination cycle is in a range from 15 minutes to 60 minutes.

Embodiments may include one or more features of the foregoing aspects, separately or in any combination, which may be combined with one or more of the following additional features, separately or in any combination.

In some embodiments, the temperature of the preset decontamination cycle is in a range from 170° F. to 190° F.

In some embodiments, the relative humidity of the preset decontamination cycle is in a range from 65% to 80%.

In some embodiments, the time of the preset decontamination cycle at the temperature and the humidity is in a range from 15 minutes to 60 minutes, more particularly from 15 minutes to 30 minutes.

In some embodiments, the preset decontamination cycle has at least the following parameters: the temperature of the preset decontamination cycle is about 185° F.; the relative humidity of the preset decontamination cycle is about 65%; and a time of the preset decontamination cycle at the temperature and the humidity is about 15 minutes.

In some embodiments, the preset decontamination cycle is performed without the use of a chemical or chemicals.

In some embodiments, the preset decontamination cycle is performed at ambient pressure.

In some embodiments, preset decontamination cycle is effective to at least provide a 3 log to 7 log reduction of MS2 Bacteriophage and/or Mycobacteria.

In some embodiments, the controller is operatively coupled to the humidity generator.

In some embodiments, the humidity generator includes a moisture source and a heating element.

In some embodiments, the moisture source includes a reservoir or a flow of water.

In some embodiments, the unit further comprising one or more sensors configured to measure the temperature and the relative humidity within the internal cavity, the one or more sensors being operatively coupled to the controller.

In some embodiments, the controller locks adjustment to the preset decontamination cycle when the decontamination cycle is running.

In some embodiments, the unit further including one or more internal supports for supporting the object to be decontaminated, wherein the one or more internal supports are adjustable based upon a size of the object or objects to be decontaminated.

In some embodiments, at least a portion of the one or more internal supports is covered with a temperature resistant coating or sleeve for restricting thermal degradation of the object to be decontaminated.

In some embodiments, the one or more supports are adapted for hanging and/or holding first responder and/or department of defense personnel gear.

In some embodiments, the one or more supports include rods or bars that span across and are supported on lateral sides of the internal cavity, wherein the rods or bars are adapted for receiving a hanger for holding first responder garments including at least jackets or vests.

In some embodiments, the housing includes wheels for facilitating portability of the decontamination unit.

In some embodiments, the contaminant is a virus, bacteria and/or spore, and wherein the preset decontamination cycle is configured to reduce a load of the contaminant at least by 3 log to 7 log reduction.

In some embodiments, the preset decontamination cycle is configured to at least provide a greater than 6 log reduction of MS2 Bacteriophage and a greater than 6 log reduction of Mycobacteria.

According to another aspect, a decontamination unit includes: a housing having an internal cavity and an opening for accessing the internal cavity; a door coupled to the housing for opening or closing the opening for permitting or restricting access to the internal cavity; a heater in thermal communication with the internal cavity and configured to heat the internal cavity; a humidity generator in fluid communication with the internal cavity and configured to supply humidity to the internal cavity; a controller configured to control a temperature and a relative humidity within the internal cavity according to a preset decontamination cycle; and supports within the internal cavity that are configured to hang service wear, in particular first responder and/or department of defense personnel gear

In some embodiments, one or more of the supports include rods that span across the internal cavity and are supported on lateral sides of the internal cavity, wherein the rods are adapted for receiving a hanger for holding first responder garments.

In some embodiments, the unit further comprising one or more additional supports within the internal cavity that are configured to support boots, gloves, or the like.

In some embodiments, the supports are adjustable.

According to another aspect, a method of decontaminating an object, includes: placing the object within an internal cavity of a decontamination unit; supplying heat to the internal cavity and increasing the temperature of the internal cavity to a temperature value in a range from 140° F. to 220° F., more particularly 170 to 200° F.; supplying humidity to the internal cavity and increasing the relative humidity of the internal cavity to a relative humidity value in a range from 50% to 100%, more particularly 50% to 80% RH; and performing decontamination at the temperature value and at the relative humidity value for a period of time in a range from 15 minutes to 60 minutes.

In some embodiments, after the period of time has lapsed, a >6 log reduction of the contaminant is achieved.

In some embodiments, the contaminant is a bacteria, virus or spore.

In some embodiments, the contaminant is a virus such as MS2 Bacteriophage (non-lipid surrogate for Sars-Cov-2) and/or a bacteria such as Mycobacteria.

In some embodiments, the object is a piece of first responder and/or department of defense personnel gear.

According to another aspect, a method of decontaminating first responder equipment includes: placing the first responder equipment within an internal cavity of a decontamination unit; supplying heat to the internal cavity; and supplying humidity to the internal cavity; wherein the supplying heat and the supplying humidity is effective to provide at least between a 3 log to 7 log reduction in bacterial or viral load on the first responder equipment.

In some embodiments, reduction in bacterial or viral load includes at least a 3 log to 7 log reduction in enveloped viruses, vegetative bacteria, fungi, non-enveloped viruses, and/or mycobacteria (Mycobacterium species).

In some embodiments, the first responder equipment includes jackets, vests, gloves, and/or helmets

Embodiments of the subject matter described in this disclosure can be implemented in combination with digital electronic circuitry, controllers, processors, computer software, firmware, and/or hardware. For example, embodiments may be implemented in a decontamination unit that uses one or more modules of computer program with instructions encoded on a non-transitory computer-readable medium for execution by, or to control the operation of, data processing apparatus. The operations may include physical manipulations of physical quantities. Usually, though not necessarily, the physical quantities take the form of electrical or magnetic signals capable of being stored, transferred, combined, compared, and otherwise manipulated in a logic and the like.

The controller may include, in addition to hardware, code that creates an execution environment for the computer program in question. The computer program (also referred to as software or code), may be deployed in any form, including as a stand-alone program or as a module, component, subroutine, or other unit suitable for use in a computing environment. The computer program may be deployed to be executed on one computer or on multiple computers that are located at one site or distributed across multiple sites and interconnected by a communication network. The processor may include all apparatus, devices, and machines suitable for the execution of a computer program, which may include, by way of example, both general and special purpose microprocessors, and any one or more processors of any kind of digital computer. Generally, the processor will receive instructions and data from a read-only memory or a random-access memory or both. The computer may include, or be operatively coupled to receive data from or transfer data to, or both, one or more mass storage devices for storing data. Devices suitable for storing computer program instructions and data include all forms of non-volatile memory, media and memory devices, including by way of example semiconductor memory devices. The processor and the memory can be supplemented by, or incorporated in, special purpose logic circuitry.

In the flow diagram(s), blocks denote “processing blocks” that may be implemented with logic. The processing blocks may represent a method step or an apparatus element for performing the method step. A flow diagram does not depict syntax for any particular programming language, methodology, or style (e.g., procedural, object-oriented). Rather, a flow diagram illustrates functional information one skilled in the art may employ to develop logic to perform the illustrated processing. It will be appreciated that in some examples, program elements like temporary variables, routine loops, and so on, are not shown. It will be further appreciated that electronic and software applications may involve dynamic and flexible processes so that the illustrated blocks can be performed in other sequences that are different from those shown or that blocks may be combined or separated into multiple components. It will be appreciated that the processes may be implemented using various programming approaches like machine language, procedural, object oriented or artificial intelligence techniques. In one example, methodologies are implemented as processor executable instructions or operations provided on a computer-readable medium. Thus, in one example, a computer-readable medium may store processor executable instructions operable to perform a method.

As used herein, an “operative connection,” or a connection by which entities are “operatively connected,” is one in which the entities are connected in such a way that the entities may perform as intended. An operative connection may be a direct connection or an indirect connection in which an intermediate entity or entities cooperate or otherwise are part of the connection or are in between the operatively connected entities. An “operative connection,” also may be one in which signals, physical communications, or logical communications may be sent or received. Typically, an operative connection includes a physical interface, an electrical interface, or a data interface, but it is to be noted that an operative connection may include differing combinations of these or other types of connections sufficient to allow operative control. For example, two entities can be operatively connected by being able to communicate signals to each other directly or through one or more intermediate entities like a processor, operating system, a logic, software, or other entity. Logical or physical communication channels can be used to create an operable connection.

It is to be understood that terms such as “top,” “bottom,” “upper,” “lower,” “left,” “right,” “front,” “rear,” “forward,” “rearward,” and the like as used herein may refer to an arbitrary frame of reference, rather than to the ordinary gravitational frame of reference.

It is to be understood that all ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural.

All ranges and ratio limits disclosed in the specification and claims may be combined in any manner. It is to be understood that unless specifically stated otherwise, references to “a,” “an,” and/or “the” may include one or more than one, and that reference to an item in the singular may also include the item in the plural.

The term “about” as used herein refers to any value which lies within the range defined by a variation of up to ±10% of the stated value, for example, ±10%, ±9%, ±8%, ±7%, ±6%, ±5%, ±4%, ±3%, ±2%, ±1%, ±0.01%, or ±0.0% of the stated value, as well as values intervening such stated values.

The phrase “and/or” should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The word “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” may refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

The phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The transitional words or phrases, such as “comprising,” “including,” “carrying,” “having,” “containing,” “involving,” “holding,” and the like, are to be understood to be open-ended, i.e., to mean including but not limited to.

Although the invention has been shown and described with respect to a certain embodiment or embodiments, it is obvious that equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

What is claimed is:

1. A decontamination unit comprising:
a housing having an internal cavity and an opening for accessing the internal cavity;
a door coupled to the housing for opening or closing the opening for permitting or restricting access to the internal cavity;
a heater in thermal communication with the internal cavity and configured to heat the internal cavity;
a humidity generator in fluid communication with the internal cavity and configured to supply humidity to the internal cavity;
a controller operatively coupled to at least the heater, the controller being configured to control a temperature and a relative humidity within the internal cavity according to a preset decontamination cycle having at least the following parameters:
the temperature of the preset decontamination cycle is in a range from 170° F. to 200° F.;
the relative humidity of the preset decontamination cycle is in a range from 50% to 80%; and
a time of the preset decontamination cycle is in a range from 15 minutes to 60 minutes.

2. The decontamination unit according to claim 1, wherein the temperature of the preset decontamination cycle is in a range from 170° F. to 190° F.

3. The decontamination unit according to claim 1, wherein the relative humidity of the preset decontamination cycle is in a range from 65% to 80%.

4. The decontamination unit according to claim 1, wherein the time of the preset decontamination cycle at the temperature and the humidity is in a range from 15 minutes to 30 minutes.

5. The decontamination unit according to claim 1, wherein the preset decontamination cycle has at least the following parameters:
the temperature of the preset decontamination cycle is about 185° F.;
the relative humidity of the preset decontamination cycle is about 65%; and
a time of the preset decontamination cycle at the temperature and the humidity is about 15 minutes.

6. The decontamination unit according to claim 1, wherein the preset decontamination cycle is performed without the use of a chemical or chemicals.

7. The decontamination unit according to claim 1, wherein the preset decontamination cycle is performed at ambient pressure.

8. The decontamination unit according to claim 1, wherein the preset decontamination cycle is effective to at least provide a 3 log to 7 log reduction of MS2 Bacteriophage and/or Mycobacteria.

9. The decontamination unit according to claim 1, wherein the controller is operatively coupled to the humidity generator.

10. The decontamination unit according to claim 1, wherein the humidity generator includes a moisture source and a heating element.

11. The decontamination unit according to claim 10, wherein the moisture source includes a reservoir or a flow of water.

12. The decontamination unit according to claim 10, further comprising one or more sensors configured to measure the temperature and the relative humidity within the internal cavity, the one or more sensors being operatively coupled to the controller.

13. The decontamination unit according to claim 1, wherein the controller locks adjustment to the preset decontamination cycle when the decontamination cycle is running.

14. The decontamination unit according to claim 1, comprising one or more internal supports for supporting the object to be decontaminated, wherein the one or more internal supports are adjustable based upon a size of the object or objects to be decontaminated.

15. The decontamination unit according to claim 14, wherein at least a portion of the one or more internal supports is covered with a temperature resistant coating or sleeve for restricting thermal degradation of the object to be decontaminated.

16. The decontamination unit according to claim 14, wherein the one or more supports include rods or bars that span across and are supported on lateral sides of the internal cavity, wherein the rods or bars are adapted for receiving a hanger for holding first responder garments including at least jackets or vests.

17. The decontamination unit according to claim 1, wherein the housing includes wheels for facilitating portability of the decontamination unit.

18-20. (canceled)

21. A decontamination unit comprising:
a housing having an internal cavity and an opening for accessing the internal cavity;
a door coupled to the housing for opening or closing the opening for permitting or restricting access to the internal cavity;
a heater in thermal communication with the internal cavity and configured to heat the internal cavity;
a humidity generator in fluid communication with the internal cavity and configured to supply humidity to the internal cavity;
a controller configured to control a temperature and a relative humidity within the internal cavity according to a preset decontamination cycle; and
supports within the internal cavity that are configured to hang service wear, wherein one or more of the supports include rods that span across the internal cavity and are supported on lateral sides of the internal cavity, wherein the rods are adapted for receiving a hanger for holding first responder garments.

22.-28. (canceled)

29. A method of decontaminating first responder equipment comprising:
placing the first responder equipment within an internal cavity of a decontamination unit;
supplying heat to the internal cavity; and
supplying humidity to the internal cavity;
wherein the supplying heat and the supplying humidity is effective to provide at least between a 3 log to 7 log reduction in bacterial or viral load on the first responder equipment.

30. The method according to claim 29,
wherein reduction in bacterial or viral load includes at least a 3 log to 7 log reduction in enveloped viruses, vegetative bacteria, fungi, non-enveloped viruses, and/or mycobacteria (Mycobacterium species); and
wherein the first responder equipment includes jackets, vests, gloves, and/or helmets.
